(19) United States
(12) Patent Application Publication
(10) Pub. No.: US 2024/0409680 A1
(43) Pub. Date: Dec. 12, 2024

(54) BIOCOMPATIBLE SELF-HEALING POLYMERS AND USES THEREOF

(71) Applicant: Technion Research and Development Foundation Limited, Haifa (IL)

(72) Inventors: Hossam HAICK, Haifa (IL); Ning TANG, Haifa (IL)

(21) Appl. No.: 18/702,135

(22) PCT Filed: Nov. 10, 2022
(86) PCT No.: PCT/IL2022/051203

§ 371 Date: Apr. 17, 2024

(30) Foreign Application Priority Data
Nov. 11, 2021 (IL) 288041

(51) Int. Cl. C08G 18/75; A61L 15/26; C08G 18/10; C08G 18/38; C08G 18/69

(52) U.S. Cl. CPC C08G 18/758; A61L 15/26; C08G 18/10; C08G 18/3855; C08G 18/69

(57) ABSTRACT

The present invention provides a self-healing biocompatible elastomer comprising polymeric chains comprising units of formula (A1), wherein R1 is selected from the group consisting of a linear (C₂-C₂₀) alkylene and —R4-S—S—R4′-, wherein R4 and R4′ are independently a linear (C₁-C₁₀) alkylene; R2 is a linear or cyclic (C₄-C₁₀) alkylene; and R3 is selected from the group consisting of a polybutadiene, a polybutene, a polyethylene, a polypropylene, and a polyisoprene, wherein the elastomer is crosslinked by hydrogen bonds between urethane linkages. Further provided is a one-pot method for preparing a self-healing biocompatible elastomer. The invention further provides an antibacterial composition and a wound dressing comprising the self-healing biocompatible elastomer. Formula (A1)

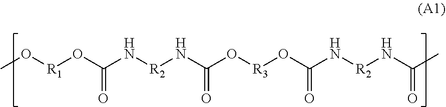

### FIELD OF THE INVENTION

The present invention is directed to biocompatible self-healing polymers, a method of preparation thereof, and uses thereof in wound dressing.

### BACKGROUND OF THE INVENTION

Various injuries (e.g., cuts, abrasions, blisters, burns, stab wound, etc.) can cause skin wounds in daily life. Intentional injuries (e.g., surgery) or unintentional injuries (e.g., abrasion) and wounds, classified as acute or chronic wounds, can compromise the integrity and protective function of the skin (Simões, Déborah, et al., European Journal of Pharmaceutics and Biopharmaceutics 127 (2018): 130-141). In clinical management, wound dressings are indispensable devices used to inhibit bacteria and promote healing by adhering to the wound. However, the need to separate the dressing from the wound during the inspection can cause secondary damage.

Wearable sensors designed to afford sensing and detecting of various physiological parameters, are particularly suitable for medical applications such as real-time diagnosis and continuous monitoring (Gao, et al., Advanced Materials 32(15) (2020): 1902133). Meanwhile, different stages of wound healing are often accompanied by changes in the physiological environment of the wound, such as, inter alia, the increase in local temperature, pH alkalization, and abnormal release of metabolites. These parameters can be considered as indicators for the assessment of the degree of wound healing. Therefore, monitoring these indicators through smart wound dressing can inform patients about the wound healing process, accurately assess wound status, and further reduce hospitalization time, prevent morbidities, and aid in therapy studies (Jankowska, et al., Biosensors and Bioelectronics 87 (2017): 312-319).

Although multiple smart patches have been developed to provide useful information of the wound healing status, most of them are not based on self-healing materials. For example, many smart wound dressings are based on hydrogels which are usually very soft so that their structure is easily deformed, and water volatility gradually affects their sensing sensitivity (Zhao et al., Advanced Functional Materials 30.17 (2020): 1910748). Furthermore, most of the current smart patches for monitoring wound status can only detect one or two wound related biomarkers (e.g., pH or/and temperature). Multi-parameter sensing that can provide a more comprehensive understanding of the wound healing status is yet to be achieved (Pang, et al. Advanced Science 7(6) (2020): 1902673; Zhu, et al., Advanced Functional Materials 30(6) (2020): 1905493). To the best of the inventors' knowledge, a smart wound dressing that can be used for wound closure to replace traditional wound stitching techniques, has not yet been reported.

Notably, wearable sensors used in wound care applications should not only have suitable flexibility to conform to human body, but should also be non-toxic and immune compatible.

WO 2020/245826 provides a solution-processable self-healing hydrolytically stable elastomer, a method for the preparation thereof, and articles of manufacture comprising the elastomer.

US 2017/0008999 is directed to a self-healing cross-linked polyurea urethane polymer and to a process for its preparation, wherein the self-healing properties of the polymer are based on the aromatic disulfide metathesis.

US 2018/0231486 pertains to a platform unit comprising a self-healing substrate comprising a dynamically crosslinked polymer comprising polymeric chains and crosslinking bridges.

US 2018/0231486 provides a self-healing platform unit for pressure and analyte sensing, and a method for fabrication thereof, the platform unit comprising a self-healing substrate comprising a dynamically crosslinked polymer comprising polymeric chains and crosslinking bridges; at least one self-healing electrode comprising a non-crosslinked polymer and metal microparticles dispersed therein, wherein the at least one self-healing electrode is deposited on the substrate; and at least one sensor comprising metal nanoparticles capped with an organic coating, wherein the at least one sensor is deposited on the substrate and is in electric contact with the at least one self-healing electrode.

WO 2013/079469 is directed to a self-healing polymer network comprising at least one polymer chain functionalized with at least two sulfur atoms in the form of thiol, thiolate or forming part of a disulfide, or a mixture thereof, wherein from 0.1-100% of the sulfur atoms are in the form of at least one transition metal thiolate, and from 99.9-0% of said sulfur atoms are in the form of thiol, a thiolate other than a transition metal thiolate, or forming part of a disulfide until completing 100% of the sulfur atoms in the form of disulfide, thiol, or thiolate, provided that if there are no cross-links in form of disulfide, then the at least one transition metal forming the transition metal thiolate is a transition metal that is able to self-assemble by metallophilic attractions.

There remains an unmet need for biocompatible, mechanically stable self-healing elastomers for use in wound dressing application, in particular in smart wound dressing applications that can monitor the wound healing process and reduce secondary injuries to wounds.

### SUMMARY OF THE INVENTION

The present invention is directed to biocompatible self-healing elastomers, which can be used in a smart multifunctional wound dressing, and methods for the preparation of said elastomers. Further provided is a wound dressing comprising said biocompatible self-healing elastomer.

While polyurethane polymers containing aromatic disulfide moieties are known to have self-healing properties, in order to use such polymers to form a self-healing wound dressing, the polymers should also be biocompatible to allow direct contact with a wound. The inventors of the present invention have surprisingly found that a polyurethane polymer having a polybutadiene backbone and aromatic disulfide moieties was not biocompatible, while aliphatic disulfide chain extender used instead of the aromatic disulfide provided the desired biocompatibility without compromising the self-healing properties of the elastomer. Thus, disclosed herein for the first time is a polyurethane-based self-healing elastomer composed of a specific combination of monomers which is biocompatible thereby being suitable for use in wound dressing applications. It has further been unexpectedly discovered that a disulfide was not required in order to provide the desired self-healing efficiency. Use of a simple alkyl chain extender in a polyurethane polymer containing a polybutadiene backbone also afforded the required self-healing efficiency and biocompatibility of the elastomer.

The present invention is further based on a surprising discovery that a film made of said biocompatible elastomers mixed with a quaternary ammonium compound, such as, for example, cetrimonium bromide (CTAB), has antibacterial properties, which are particularly beneficial in the wound dressing application. It has been further found by the inventors that addition of 1% (w/w) of CTAB to the self-healing elastomer imparts the antibacterial properties to the elastomer without affecting its biocompatibility and/or self-healing properties.

The inventors have used a convenient one-pot synthesis process, which allows to control the molar ratio between the monomers and obtain the desired polymer structure. The inventors have further used the synthesized elastomers in the preparation of a multifunctional wound dressing. The dressing comprises said biocompatible self-healing elastomer as a substrate, and at least one sensor configured to detect at least one parameter of the wound. Such wound dressing can be designed to have multiple sensing capabilities, including, inter alia, monitoring of pH, temperature, glucose, and/or uric acid through a sensing layer. Such wound dressing can also include a drug-releasing layer, adapted for controlled release of a suitable drug based on feedback signals from the wound dressing sensors. In addition, said wound dressing, which is based on a biocompatible self-healing elastomer which preferably has antibacterial properties, can be used in a wound closure to reconnect wound skin instead of traditional stitching techniques. The wound dressing can be further used in surgery, for example, by applying the dressing to the intended site of an incision and performing the incision atop the dressing, such that the separated parts of the dressing can be connected to assist in the healing of the incision.

According to one aspect, the present invention provides a self-healing biocompatible elastomer comprising polymeric chains comprising units of formula (A1):

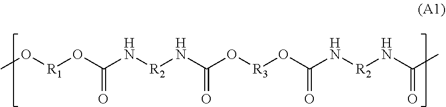

wherein R₁ is selected from the group consisting of a linear (C₂-C₂₀)alkylene and R₄—S—S—R₄′—, wherein R₄ and R₄′ are each independently a linear (C₁-C₁₀)alkylene; R₂ is a linear or cyclic (C₄-C₁₀)alkylene; and R₃ is selected from the group consisting of a polybutadiene, a polybutene, a polyethylene, a polypropylene, and a polyisoprene.

According to some embodiments, R₁ is a linear (C₂-C₂₀)alkylene. In certain embodiments, R₁ is a linear C₁₀ alkylene.

According to some embodiments, R₁ is R₄—S—S—R₄′—. In certain embodiments, each one of R₄ and R₄′ is a C₂ alkylene.

According to some embodiments, the S—S content of the elastomer is up to about 3% (w/w).

According to some embodiments, R₂ is selected from the group consisting of butylene, hexylene, cyclohexylene, and decylene. Each possibility represents a separate embodiment.

According to some embodiments, R₂ is 3-methylene-3,5,5-trimethyl-1-cyclohexyl, also referred to herein as 1,1,3,3-tetramethyl cyclohexyl (D1):

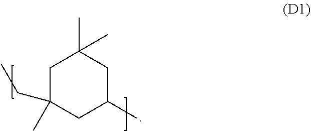

According to some embodiments, R₃ is a polybutadiene. According to additional embodiments, the polybutadiene comprises 1,3-butadiene derived-monomer units of formula (B1), formula (B2), and formula (B3),

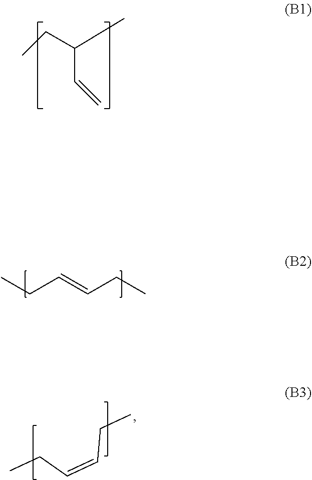

wherein the proportion of the monomer unit of formula (B1) is 10 to 60 mole percent, the proportion of the monomer unit of formula (B2) is 20 to 70 mole percent, and the proportion of the monomer unit of formula (B3) is 10 to 50 mole percent in the entirety of the 1,3-butadiene-derived monomer units present in one unit of formula (A1).

According to some embodiments, the polybutadiene comprises about 20 mole percent monomer units of formula (B1), 60 mole percent monomer units of formula (B2), and 20 mole percent monomer units of formula (B3) in the entirety of the 1,3-butadiene-derived monomer units present in one unit of formula (A1).

According to some embodiments, the unit of formula (A1) is:

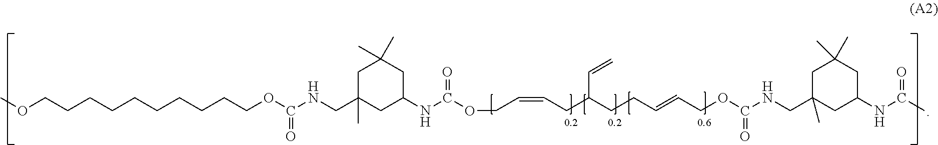

According to some embodiments, the elastomer has the structure of formula (A3):

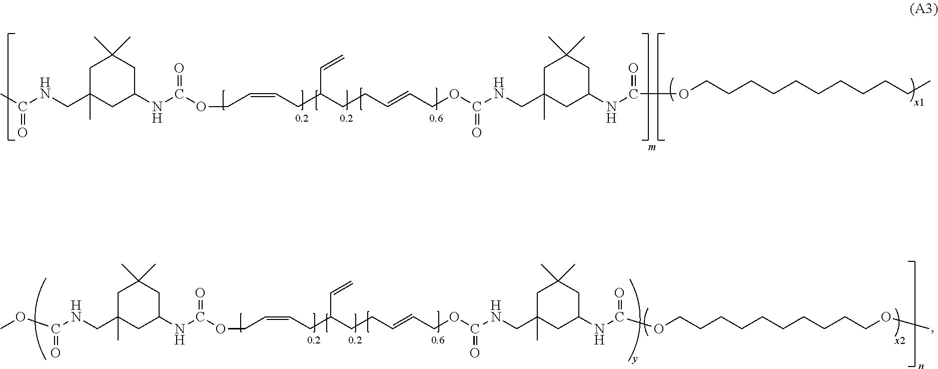

wherein 0≤y<(x1+x2).

According to some embodiments, m ranges between 1 and 1000, n ranges between 1 and 1000, y ranges between 1 and 100, x1 ranges between 1 and 100, and x2 ranges between 1 and 100, including each integer within the specified range.

According to some embodiments, the unit of formula (A1) is:

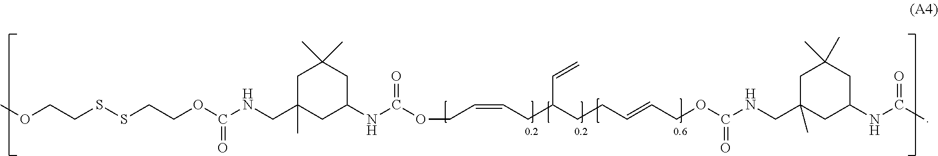

According to some embodiments, the elastomer has the structure of formula (A5):

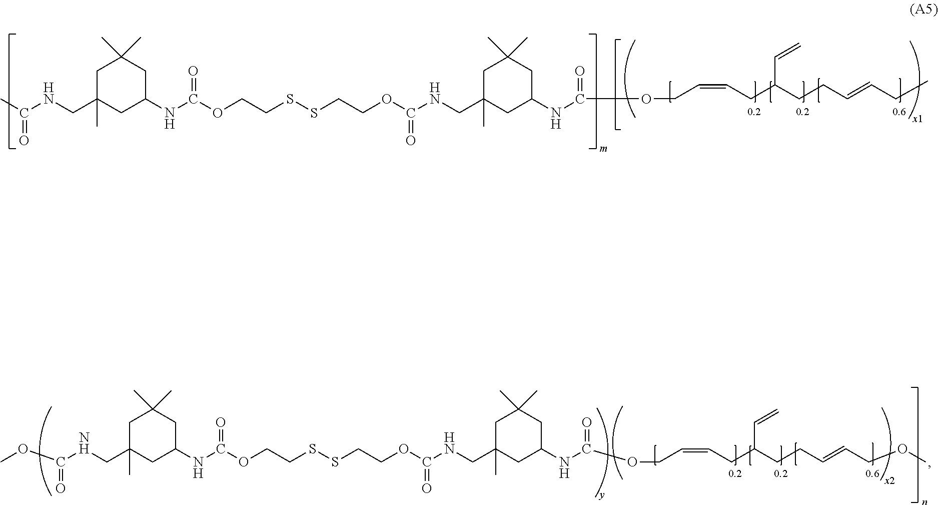

wherein 0≤y<(x1+x2).

According to some embodiments, m ranges between 1 and 1000, n ranges between 1 and 1000, y ranges between 1 and 100, x1 ranges between 1 and 100, and x2 ranges between 1 and 100, including each integer within the specified range.

In another aspect, the present invention provides a one-pot method for preparing a self-healing biocompatible elastomer, the method comprising reacting a hydroxyl-terminated polybutadiene (HTPB) with a linear or cyclic (C₄-C₁₀)alkylene diisocyanate compound and a hydroxyl-terminated compound selected from a linear (C₂-C₂₀)diol and a hydroxyl-terminated linear (C₁-C₁₀)alkyl disulfide.

According to some embodiments, the hydroxyl-terminated compound is a linear (C₂-C₂₀)diol.

According to some embodiments, the hydroxyl-terminated compound is 1,10-decanediol.

According to some embodiments, the hydroxyl-terminated compound is a hydroxyl-terminated linear (C₁-C₁₀)alkyl disulfide.

According to some embodiments, the hydroxyl-terminated compound is 2-hydroxyethyl disulfide.

According to some embodiments, the diisocyanate compound is selected from the group consisting of isophorone diisocyanate (IPDI), 1,4-tetramethylene diisocyanate, 1,6-hexamethylene diisocyanate (HDI), and 1,10-decamethylene diisocyanate. Each possibility represents a separate embodiment. According to certain embodiments, the diisocyanate compound is IPDI.

According to some embodiments, the HTPB comprises 1,3-butadiene derived-monomer units of formula (B1), formula (B2), and formula (B3), wherein the proportion of the monomer unit of formula (B1) is 10 to 60 mole percent, the proportion of the monomer unit of formula (B2) is 20 to 70 mole percent, and the proportion of the monomer unit of formula (B3) is 10 to 50 mole percent in the entirety of the 1,3-butadiene-derived monomer units present in the HTPB. In certain embodiments, the HTPB comprises about 20 mole percent monomer units of formula (B1), 60 mole percent monomer units of formula (B2), and 20 mole percent monomer units of formula (B3).

According to some embodiments, the molar ratio between the HTPB, the hydroxyl-terminated compound, and the linear or cyclic (C₄-C₁₀)alkylene diisocyanate compound is about 1:1:2.1.

In yet another aspect, the present invention provides an elastomer obtained by the method according to the aspect and various embodiments hereinabove.

In still another aspect, the present invention provides an antibacterial composition comprising the elastomer according to the various aspects and embodiments hereinabove, and a quaternary ammonium compound.

According to some embodiments, the quaternary ammonium compound is cetyltrimethylammonium bromide (CTAB).

According to some embodiments, the quaternary ammonium compound is present in the composition in a weight percentage of up to about 1% of the total weight of the composition.

According to some embodiments, the antibacterial composition is in a form of a film.

In yet another aspect, the present invention provides a method for the preparation of an antibacterial composition comprising mixing the elastomer according to the various aspects and embodiments hereinabove, the quaternary ammonium compound and a solvent to form a homogeneous mixture, and evaporating the solvent.

In yet another aspect, the present invention provides a wound dressing comprising a film made of the elastomer or the antibacterial composition according to the various aspects and embodiments hereinabove.

According to some embodiments, the wound dressing comprises at least one sensor for the detection of one or more parameters of the wound, wherein the at least one sensor is embedded within or deposited onto the film. In certain embodiments, the at least one sensor is selected from the group consisting of a glucose sensor, a pH sensor, and a temperature sensor. Each possibility represents a separate embodiment.

According to some embodiments, the at least one sensor comprises an electrode and a sensing layer disposed on a portion of said electrode and/or electrically connected thereto, and optionally, a reference electrode. In certain embodiments, the electrode is made of a micro-sized or nanosized conductive material embedded within or deposited onto the film.

According to some embodiments, the conductive material is selected from the group consisting of a metal, a metal alloy, a metal carbide, a metal nitride, a metal oxide, a metal silicide, carbon, a polymer, ceramics, and combinations thereof and/or wherein the conductive material has a form selected from the group consisting of nanoparticles, nanowires, nanotubes, nanoflakes, nanofibers, nanoribbons, nano-whiskers, nanostrips, nanorods, and combinations thereof. Each possibility represents a separate embodiment.

According to some embodiments, the sensing layer comprises a material selected from the group consisting of a biorecognition element, a redox-active element, an electrically conducting material, a thermally conductive material, and any combination thereof. Each possibility represents a separate embodiment.

According to some embodiments, the sensing layer comprises a material selected from the group consisting of polyethyleneimine (PEI), glucose oxidase (GOx), carbon nanotubes, reduced graphene oxide (rGO), polyaniline (PANI), K₃[Fe(CN)₆](Prussian blue), and any combination thereof. Each possibility represents a separate embodiment. In certain embodiments, the wound dressing comprises a glucose sensor comprising an electrode made of Ag nanowires and a sensing layer comprising Prussian blue and glucose oxidase; a pH sensor comprising an electrode made of Ag nanowires and a sensing layer comprising PANI; and a temperature sensor comprising an electrode made of Ag nanowires and a sensing layer comprising PEI and reduced graphene oxide.

According to some embodiments, the wound dressing further comprises an additional film made of the elastomer or the antibacterial composition according to the various aspects and embodiments hereinabove, wherein the additional film covers at least a portion of the at least one sensor.

According to some embodiments, the wound dressing further comprises at least one of a drug release layer, a self-cleaning protecting layer, and a wearable data processing device. Each possibility represents a separate embodiment.

According to some embodiments, the wound dressing is for use in the treatment and/or monitoring a condition of a wound. In certain embodiments, the condition of the wound is monitored by the at least one sensor.

According to some embodiments, the wound dressing is for use in performing a surgical incision on a body part, wherein the wound dressing is applied to said body part and the incision is performed atop the wound dressing.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

FIG. 1 is a scheme demonstrating the synthetic procedure of an antibacterial composition comprising a polyurethane-polybutadiene elastomer comprising 1,10-decanediol as a chain extender and CTAB (PUIDE-CTAB).

FIG. 2 is a scheme demonstrating the structures and proposed self-healing mechanism of PUIDE-CTAB based on the reversible hydrogen bonds.

FIG. 3 shows the structure and corresponding ¹H-NMR spectrum of PUIDE-CATB.

FIG. 4 shows the FTIR spectrum of PUIDE-CTAB. The characteristic peak assigned to the H-bonded C═O groups (1679 cm⁻¹) is depicted.

FIG. 5 shows a TGA analysis of PUIDE-CTAB at 20° C./min under air atmosphere.

FIG. 6 is an image demonstrating the contact angle of water on a PUIDE-CTAB film.

FIG. 7 is an image demonstrating the contact angle of water on a different PUIDE sample.

FIG. 8 shows the transmittance spectrum of a PUIDE-CTAB sample (40×30×0.5 mm). The inset photograph demonstrates the transparency of the film.

FIG. 9 is a scheme demonstrating the proposed ideal structure and self-healing mechanism of a polyurethane-polybutadiene elastomer comprising 2-hydroxyethyl disulfide as a chain extender (PUIDS) network based on reversible disulfide and hydrogen bonds.

FIG. 10 shows tensile Strain-Stress curves of pristine and notched PUIDE-CTAB samples (gauge length: 15 mm; width: 2 mm; thickness: 0.45-0.55 mm).

FIG. 11 shows Strain-Stress curves of PUIDE-CTAB films at different loading rates.

FIGS. 12A-12B are images demonstrating a notched PUIDE-CTAB film before stretch (12A) and at a certain strain (12B).

FIG. 13 shows the fatigue resistance of PUIDE-CTAB cyclic Stress-Strain curves over 5 cycles of successive loading/unloading processes without rest, and the 6^{th} cycle after resting at room temperature for 1 h (relaxed for 1 hour).

FIG. 14 shows the fatigue resistance of PUIDE-CATB samples with 1000% strain.

FIG. 15 shows the self-recovery of the loading/unloading curves of PUIDE-CTAB.

FIG. 16 shows the self-recovery of PUIDE-CATB samples with 1000% strain.

FIGS. 17A-17B show optical images demonstrating the self-healing process of the scratched PUIDE-CTAB film at 40° C. for 2 h. Scale bar: 100 m.

FIG. 18 demonstrates the self-healing ability and recovery of stretchability after a complete cut (top) and a photograph of PUIDE-CTAB film after a complete cut before being healed for 30 min (bottom).

FIG. 19 shows Stress-Strain curves of original and full-cut PUIDE-CTAB samples healing at 25° C. (RT) for different periods of time, and at 40-50° C. for 6 h. In all the experiments, the stretching rate was 100 mm min⁻¹.

FIG. 20 shows a column chart representing the healing efficiencies of PUIDE-CTAB samples under different healing times and conditions.

FIG. 21 shows the dynamic mechanical analysis results of the storage modulus (E′) and loss modulus (E″) at different frequencies.

FIG. 22 shows the dynamic mechanical analysis results of the storage modulus (E′) and loss modulus (E″) versus temperature.

FIG. 23 shows typical Strain-Stress curves of original PUIDS samples.

FIG. 24 shows typical tensile Stress-Strain curves of original and notched antibacterial films comprising a mixture of PUIDS and 1% (w/w) CTAB (PUIDS-1-CTAB1%) samples (gauge length: 16 mm; width: 2 mm; thickness: 0.45-0.55 mm).

FIGS. 25A-25B show optical images of the scratched PUIDS-1-CTAB1% in the self-healing process at t=0 (25A) and after 2 hours at 60° C. (25B), scale bar: 100 m.

FIG. 26 shows photographs of the polymer after being cut into two pieces and then healed for 30 min at room temperature (top) and stretched (bottom).

FIG. 27 shows Stress-Strain curves of original and full-cut PUIDS-1-CTAB1% samples healing at 25° C. (RT) for different time periods and at 40-60° C. for 6 h.

FIG. 28 shows the fatigue resistance of PUIDS-1-CTAB1%; Cyclic Stress-Strain curves of 11 times successive loading/unloading processes of polymer without rest (origin and cycles 1-10), and the 12^{th} loading/unloading cycle after resting at room temperature for 30 min (relaxed for 30 min).

FIG. 29 shows the self-recovery of the loading/unloading curves of PUIDS-1-CTAB1%.

FIG. 30 shows live/dead H&E staining images of L02 cells after being co-cultured with PUIDE-CTAB for 48 h. Scale bar, 200 μm.

FIG. 31 shows the hemolysis evaluation after red blood cells had been incubated with different amounts of PUIDE-CTAB for 2 h, using PBS buffer and deionized water as positive (P) and negative (N) controls, respectively. The inset shows a photo of the hemolysis after centrifugation (n=4).

FIGS. 32A-32B show photographs of (32A) the PUIDE-CTAB sample incubated under the mouse skin, and (32B) the state of the polymer after 10 days.

FIGS. 33A-33H show the effect of PUIDE-CTAB on the hematopoietic and metabolic systems of mice. PUIDE-CTAB samples (20×15×0.5 mm) were implanted under the skin for 10 days, and their hematopoietic system and metabolite concentrations were compared to that of untreated mice (control) (n=3).

FIGS. 34A-34D show the effect of PUIDE-CTAB on the hematopoietic and metabolic systems of mice. PUIDE-CATB samples (20×15×0.5 mm) were implanted under the skin of rats for 10 days, and their hematopoietic system and metabolite concentrations were compared to that of the mice without treatment (control) (n=3).

FIG. 35 shows H&E staining images of the major organs of mice treated with PUIDE-CTAB on the 10^{th} day. Scale bar, 200 μm.

FIG. 36 shows the cell toxicity evaluation of PUIDS-1-CTAB1% on human L02 cells for 24 h and 48 h at different concentrations, n=3.

FIG. 37 shows the hemolysis evaluation of the mouse red blood cells incubated with various concentrations of PUIDS-1-CTAB1% for 2 h. Positive control (P): H₂O; Negative control (N): PBS buffer. The inset shows a photo of the hemolysis of different samples after centrifugation.

FIG. 38 shows in-vivo biocompatibility tests of PUIDS-1-CTAB1%. H&E staining of major organs from a mouse model following treatment with PUIDS-1-CTAB1% on the 7^{th} day. Scale bar, 200 m.

FIG. 39 shows a photo of bacterial suspensions extracted from cultured tube in which the E. coli suspensions were co-cultured with PUIDE and PUIDE-CTAB samples. The nutrient solution and bacterial suspension cultured without polymers, as blank group and control group, respectively, were placed in the same conditions as the above bacterial suspensions cultured with different polymers.

FIG. 40 shows the bactericidal rate of PUIDE-CTAB to E. coli and S. aureus under different treatment intervals, n=3.

FIG. 41 shows confocal microscopy micrographs of live/dead staining to assess bacterial viability of E. coli after being co-cultured without/with PUIDE-CTAB for 6 h. Scale bar, 20 m.

FIG. 42 shows confocal microscopy micrographs of live/dead staining to assess bacterial viability of S. aureus after being co-cultured without/with PUIDE-CTAB for 6 h. Scale bar, 20 m.

FIGS. 43A-43B show SEM images with E. coli (43A) untreated, and (43B) treated with PUIDE-CTAB.

FIGS. 44A-44B show SEM images with S. aureus (44A) untreated, and (44B) treated with PUIDE-CTAB.

FIG. 45 shows a column chart demonstrating the optical density value (ODV, 630 nm) of E. coli suspensions extracted from the culture tube with different treatments after 24 h.

FIG. 46 shows macroscopic clinical photos of the wounds in mouse models on the 3^{rd}, 5^{th} and 7^{th} day of different treatments. Scale bar, 0.5 cm.

FIG. 47 shows macroscopic clinical photos of the wounds in a mouse model on day 1. Scale bar, 0.5 cm.

FIG. 48 shows a column chart demonstrating the statistical comparison of the wound area in each group at the 3^{rd}, 5^{th} and 7^{th} day, n=5. *p<0.05, **p<0.01, ***p<0.001.

FIG. 49 shows H&E staining and Masson's trichrome staining of wound sites at the 3^{rd}, and 7^{th} day. Scale bar, 200 m.

FIG. 50 shows immunocytochemistry images demonstrating the expression levels of cell nucleus (4′,6-diamidino-2-phenylindole (DAPI) staining) and VEGF in wound tissues under different treatments on the 7^{th} day. Scale bar, 100 m.

FIGS. 51A-51B show FACS analysis. (51A) FACS of neutrophil by staining with anti-Ly6G/6C antibody in mouse blood under different treatments on the 7^{th} day, and (51B) a column chart demonstrating a comparison of the statistical results of Ly6G/6C positive cells in blood, n=3. **p<0.01.

FIG. 52 shows a schematic diagram of the PUIDE-CTAB.

FIG. 53 shows a photo of the sensing part of MFDW with 3 different sensors: glucose (PB/GOx), pH (PANI), and temperature (PEI/rGO). Scale bar: 1 cm.

FIG. 54 shows the selective functionalization process of the sensor array in a multifunctional wound dressing system.

FIGS. 55A-55C show CV plots of electrodes (55A) during the PANI electrodeposition process, (55B) during the PB electrodeposition process, and (55C) in PBS after the electrodeposition of PB.

FIG. 56 shows the chronoamperometric response to the glucose concentration ranging from 0 to 4 mM.

FIG. 57 shows the relationship between glucose concentration and response current.

FIG. 58 shows a calibration plot of generated current versus glucose concentrations, with a linear response (r²=0.968) between 0 and 200 μM and with an average sensitivity of 1.72 μA/mM.

FIG. 59 shows the performance of the glucose sensor.

FIG. 60 shows the response of the glucose sensor to 400 μM glucose in the presence of common electroactive interferents found in wound fluid.

FIG. 61 shows the long-term stability of the glucose sensor.

FIG. 62 shows the measurements of the real-time response to pH levels in the range of 4-10.

FIG. 63 shows pH sensing data with a linear response (r²=0.991), and an average sensitivity of −30.8 mV/pH.

FIG. 64 shows the effect of volume on pH sensors.

FIG. 65 shows the pH dependency of glucose sensors.

FIG. 66 shows the real-time response of the temperature sensors.

FIG. 67 shows temperature sensing data with a linear response (r²=0.994), and an average sensitivity of ˜0.54%/° C.

FIG. 68 shows the repeatability of the temperature sensors.

FIG. 69 shows the dynamic response of the resistance changes under increased temperature.

FIGS. 70A-70B show the effects of (70A) temperature on a glucose sensor, and (70B) temperature on a pH sensor.

FIG. 71 shows a photograph demonstrating a mouse with multifunctional wound dressing for sutureless wound closure and wound monitoring.

FIG. 72 shows representative images of tape-, suture-, and MFWD-treated incisions at days 3, 5, 7 and 9 after surgery. Scale bar, 0.5 cm.

FIG. 73 shows representative images of tape-, suture-, and MFWD-treated incisions at day 1. Scale bar, 0.5 cm.

FIG. 74 shows a column chart demonstrating the breaking strengths of mouse skin incisions after different treatments (n=3).

FIG. 75 shows H&E staining and Masson's trichrome staining of wound sites after 7 days treatment. Scale bar, 200 m.

FIG. 76 shows continuous monitoring of the glucose in the wounds with/without bacterial infection during the healing process.

FIG. 77 shows continuous monitoring of the pH of the wounds with/without bacterial infection during the healing process.

FIG. 78 shows continuous monitoring of the temperature of the wounds with/without bacterial infection during the healing process.

FIG. 79 shows the chemical structure of a self-healing polybutadiene polyurea urethane elastomer based on an aromatic disulfide dynamic linkage (PBPUU) and a scheme demonstrating the synthetic procedure used for its preparation.

FIG. 80 shows photographs of the PBPUU demonstrating the self-healing ability and the recovery of stretchability after a complete cut.

### DETAILED DESCRIPTION OF THE INVENTION

The present invention is directed to a biocompatible self-healing elastomer which can be used as a wound dressing to promote sutureless wound closure, as well as a smart wound dressing equipped with a sensing layer, to allow real-time monitoring of the healing status, and, optionally, a drug-releasing layer, to allow timely therapeutic intervention.

The inventors of the present invention have developed a novel self-healing elastomer-based multifunctional wound dressing (MFWD) integrated with a sensing system for sutureless wound closure and wound status monitoring. The self-healing elastomer on which the MFWD is based has excellent mechanical robustness, flexibility and biocompatibility. The elastomer can be mixed with an antiseptic agent, such as, e.g., a quaternary ammonium compound, and cast as a self-healing mechanically stable film, wherein the antiseptic agent imparts antibacterial properties to the entire film. In vivo animal studies demonstrated that, owing to the excellent self-healing property of the elastomer, MFWD can contract the wound edges by mechanical force to achieve effectively sutureless wound closure. With the help of an integrated sensing system, MFWD can comprehensively report on the wound status by monitoring, inter alia, the temperature, pH, glucose, and/or uric acid concentrations to ensure proper wound recovery conditions. The biocompatible self-healing elastomer-based MFWD therefore offers great advantage in wound management applications, through non-invasive wound closure and wound healing monitoring.

According to one aspect, the present invention provides a self-healing biocompatible elastomer comprising polymeric chains comprising units of formula (A1)

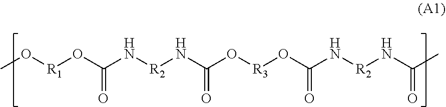

wherein
R₁ is selected from the group consisting of a linear (C₂-C₂₀)alkylene and R₄—S—S—R₄′—, wherein R₄ and R₄′ are independently a linear (C₁-C₁₀)alkylene;
R₂ is a linear or cyclic (C₄-C₁₀)alkylene; and
R₃ is selected from the group consisting of a polybutadiene, a polybutene, a polyethylene, a polypropylene, and a polyisoprene.

The term “elastomer”, as used herein, refers to a polymeric material which exhibits a combination of high elongation or extensibility, high retractability to its original shape or dimensions after removal of the stress or load, with little or no plastic deformation. The elastomer, according to the principles of the present invention, possesses a low modulus and it can be stretched by applying a low load. The terms “polymeric material” and “polymer” refer to a macromolecule composed of multiple repeated subunits, known as monomers. Polymers, both natural and synthetic, are produced via polymerization of a plurality of monomers. The polymer is composed of polymer chains, said chains being linear or branched.

The term “self-healing”, as used herein, refers in some embodiments to the ability of the elastomer to physically recombine following mechanical damage. The recombination can include, but is not limited to, spontaneous recombination, magnetic recombination, and repair agent recombination. Each possibility represents a separate embodiment. The term “mechanical damage”, as used herein, refers to a partial or full disassociation between two parts of the elastomer. Mechanical damage applied to the elastomer may include, inter alia, a scratch, a partial cut or a full cut. Each possibility represents a separate embodiment. The term “scratch”, as used herein refers to a disassociation depth of up to about 10% of the elastomer thickness. The term “partial cut”, as used herein refers to a disassociation depth of above about 10% but less than 100% of the elastomer thickness. The term “full cut”, as used herein refers to a disassociation depth of 100% of the elastomer thickness. Mechanical damage can include multiple cycles of mechanical damage.

The elastomer can be crosslinked by at least one of hydrogen bonds, disulfide bonds, and metal coordination bonds. Each possibility represents a separate embodiment.

The terms “crosslinked” and “crosslinking”, as used herein, refer to covalent bonds, hydrogen bonds and/or coordination bonds formed between the polymeric chains of the elastomer.

In some embodiments, the elastomer is dynamically crosslinked by hydrogen bonds between urethane linkages. The terms “dynamically crosslinked” and “dynamic crosslinking”, as used herein, refer to covalent bonds and/or hydrogen bonds formed between the polymeric chains of the elastomer, which can be cleaved and spontaneously reformed. In some embodiments, the elastomer is dynamically crosslinked by disulfide bonds.

The term “alkyl” refers to a saturated aliphatic hydrocarbon, including straight-chain (also termed “linear alkyl”), branched-chain and cyclic alkyl groups. As used herein, affixing the suffix “-ene” to a group indicates that the group is a divalent moiety, e.g., alkylene is the divalent moiety of alkyl.

According to some embodiments, the polymeric chains comprise from about 10 to about 10,000 units of formula (A1) per chain, including each integer within the specified range. In some embodiments, said chains are dynamically crosslinked by hydrogen bonds between the urethane linkages groups.

The polymeric chains can contain additional units, which differ from the units of formula A1 in the number of times R₁ appears within the unit. For example, the polymeric chains may contain additional units which include two, three, four, five or more of R₁ within the unit.

According to some embodiments, the elastomer has a molecular weight ranging from about 1000 g/mole to about 10,000 g/mole, including each value within the specified range.

In some embodiments, R₁ is a linear (C₂-C₂₀)alkylene. In further embodiments, R₁ is a linear (C₄-C₁₆)alkylene. In yet further embodiments, R₁ is a linear (C₈-C₁₂)alkylene. In certain embodiments, R₁ is a linear C₁₀ alkylene.

In some embodiments, R₁ is R₄—S—S—R₄′—. In further embodiments, each one of R₄ and R₄′ are individually a linear (C₁-C₁₀)alkylene. In still further embodiments, each one of R₄ and R₄′ are individually a linear (C₁-C₈)alkylene. In yet further embodiments, each one of R₄ and R₄′ are individually a linear (C₁-C₆)alkylene. In still further embodiments, each one of R₄ and R₄′ are individually a linear (C₁-C₄)alkylene. In certain embodiments, each one of R₄ and R₄′ is a C₂ alkylene, i.e. ethylene.

The elastomer can have varying contents of the S—S moieties. In some embodiments, the S—S content of the elastomer ranges from about 1 to about 6% (w/w), including each value within the specified range. In some embodiments, the S—S content of the elastomer is up to about 3% (w/w).

The term “S—S content”, as used herein, refers to the weight percentage of the S—S moieties of the total weight of the elastomer.

R₂ can be a linear or cyclic alkylene. According to some embodiments, R₂ is selected from the group consisting of butylene, hexylene, cyclohexylene, and decylene. Each possibility represents a separate embodiment of the invention. In certain embodiments, R₂ is 3-methylene-3,5,5-trimethyl-1-cyclohexyl, also denoted 1,1,3,3-tetramethyl cyclohexyl represented by the structure of formula (D1)

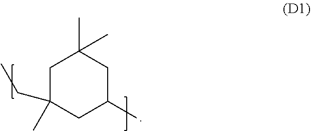

In some embodiments, R₃ is a polyolefin including, but not limited to, polybutene, polyethylene, polypropylene, and the like. Each possibility represents a separate embodiment.

In some embodiments, R₃ is a polyisoprene or a polybutadiene. Each possibility represents a separate embodiment.

In currently preferred embodiments, R₃ is a polybutadiene. In additional embodiments, the polybutadiene comprises 1,3-butadiene derived-monomer units of formula (B1), formula (B2), and formula (B3),

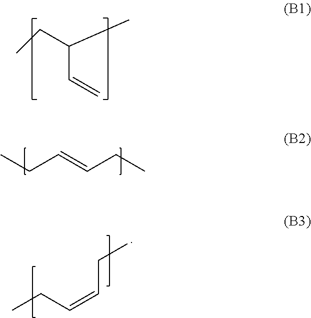

In some embodiments, the proportion of the monomer unit of formula (B1) in the entirety of the 1,3-butadiene-derived monomer units present in one unit of formula (A1) is 10 to 60 mole percent, including each value within the specified range. In further embodiments, the proportion of the monomer unit of formula (B1) in the entirety of the 1,3-butadiene-derived monomer units present in one unit of formula (A1) is 10 to 30 mole percent, including each value within the specified range.

In some embodiments, the proportion of the monomer unit of formula (B2) in the entirety of the 1,3-butadiene-derived monomer units present in one unit of formula (A1) is 20 to 70 mole percent, including each value within the specified range. In further embodiments, the proportion of the monomer unit of formula (B2) in the entirety of the 1,3-butadiene-derived monomer units present in one unit of formula (A1) is 50 to 70 mole percent, including each value within the specified range.

In some embodiments, the proportion of the monomer unit of formula (B3) in the entirety of the 1,3-butadiene-derived monomer units present in one unit of formula (A1) is 10 to 50 mole percent, including each value within the specified range. In further embodiments, the proportion of the monomer unit of formula (B3) in the entirety of the 1,3-butadiene-derived monomer units present in one unit of formula (A1) is 10 to 30 mole percent, including each value within the specified range.

According to some exemplary embodiments, the polybutadiene-containing polyurethane comprises about 20 mole percent monomer units of formula (B1), 60 mole percent monomer units of formula (B2), and 20 mole percent monomer units of formula (B3) of the entirety of the 1,3-butadiene-derived monomer units present in one unit of formula (A1).

Some of the 1,3-butadiene derived-monomer units in the polybutadiene can be saturated. Accordingly, in certain embodiments, the polybutadiene comprises from about 0 to about 80 mole percent of 1,3-butadiene derived-monomer units in which the C═C double bond is hydrogenated. In further embodiments, the polybutadiene comprises from about 10 to about 70 mole percent of 1,3-butadiene derived-monomer units in which the C═C double bond is hydrogenated. In still further embodiments, the polybutadiene comprises from about 20 to about 60 mole percent of 1,3-butadiene derived-monomer units in which the C═C double bond is hydrogenated. In yet further embodiments, the polybutadiene comprises from about 30 to about 50 mole percent of 1,3-butadiene derived-monomer units in which the C═C double bond is hydrogenated.

In some exemplary embodiments, the unit of formula (A1) is represented by the structure of formula (A2):

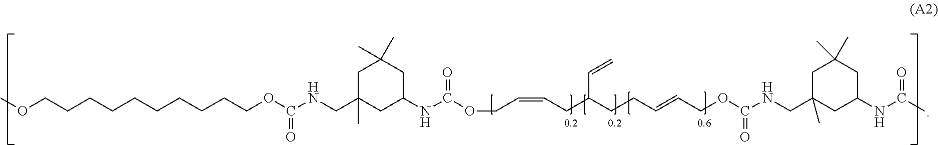

In some related embodiments, the elastomer has the structure of formula (A3):

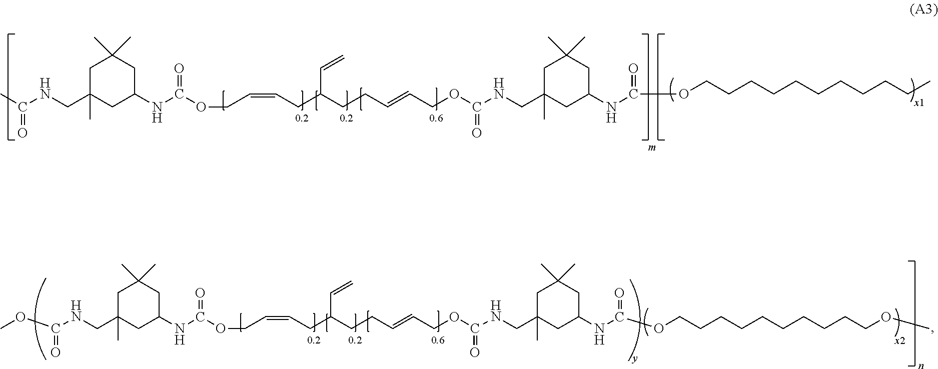

wherein 0≤y<(x1+x2).

In further embodiments, m ranges between 1 and 1000, n ranges between 1 and 1000, y ranges between 1 and 100, x1 ranges between 1 and 100, and x2 ranges between 1 and 100, including each integer within the specified ranges.

In some exemplary embodiments, the unit of formula (A1) is represented by the structure of formula (A4):

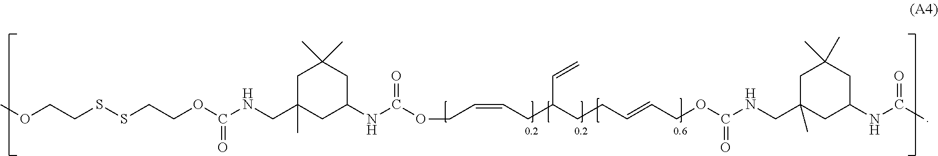

In some related embodiments, the elastomer has the structure of formula (A5):

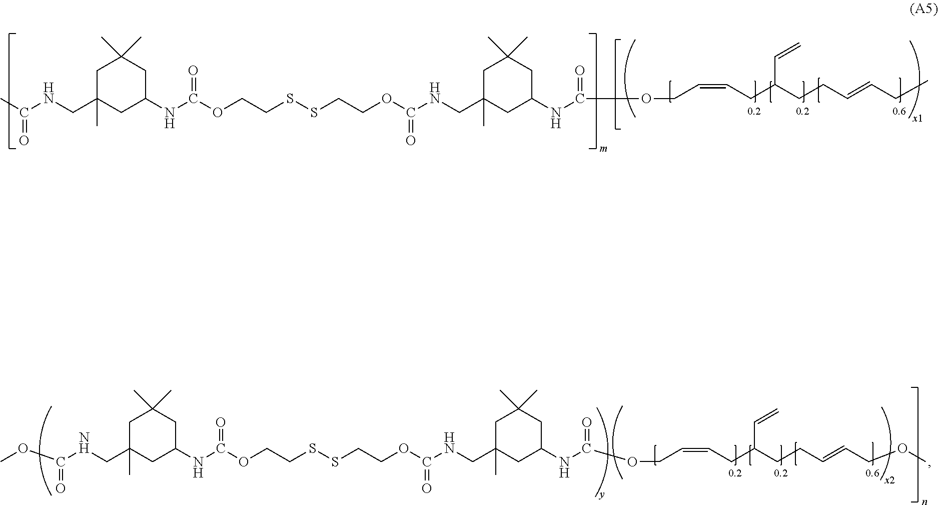

wherein 0≤y<(x1+x2).

In further embodiments, m ranges between 1 and 1000, n ranges between 1 and 1000, y ranges between 1 and 100, x1 ranges between 1 and 100, and x2 ranges between 1 and 100, including each integer within the specified ranges.

In another aspect, there is provided an elastomer composed of: a hydroxyl-terminated polybutadiene (HTPB), a (C₄-C₁₀)alkylene diisocyanate compound and a hydroxyl-terminated compound selected from a linear (C₂-C₂₀)diol and a hydroxyl-terminated linear (C₁-C₁₀)alkyl disulfide.

In yet another aspect, there is provided a one-pot method for preparing a self-healing biocompatible elastomer, the method comprising reacting a hydroxyl-terminated polybutadiene (HTPB) with a linear or cyclic (C₄-C₁₀)alkylene diisocyanate compound and a hydroxyl-terminated compound selected from a linear (C₂-C₂₀)diol and a hydroxyl-terminated linear (C₁-C₁₀)alkyl disulfide.

The term “diol”, as used herein, refers to any organic compound in which the two hydroxyl functional groups (—OH) are bound to carbon atoms. The term “linear (C₂-C₂₀)diol”, as used herein, refers to a diol having an aliphatic linear hydrocarbon chain with 2-20 carbons.

The term “disulfide”, as used herein, refers to a pair of sulfur atoms having the structure of R—S—S—R′, wherein R and R′ may be the same or different with each possibility representing a separate embodiment. The term “hydroxyl-terminated linear (C₁-C₁₀)alkyl disulfide”, as used herein, refers to a disulfide compound having an aliphatic linear hydrocarbon chain with 1-10 carbons attached to each sulfur atom, wherein the compound has two terminal OH groups.

In some embodiments, the hydroxyl-terminated compound is a linear (C₂-C₂₀)diol. In further embodiments, the hydroxyl-terminated compound is a linear (C₄-C₁₆)diol. In yet further embodiments, the hydroxyl-terminated compound is a linear (C₆-C₁₄)diol. In certain embodiments, the hydroxyl-terminated compound is a linear C₁₀ diol. In some exemplary embodiments, the hydroxyl-terminated compound is 1,10-decanediol.

In some embodiments, the hydroxyl-terminated compound is a hydroxyl-terminated linear (C₁-C₁₀)alkyl disulfide, also referred to herein as linear (C₁-C₁₀)alkyl disulfide having hydroxy termini. In further embodiments, the hydroxyl-terminated compound is a hydroxyl-terminated linear (C₁-C₅)alkyl disulfide. In yet further embodiments, the hydroxyl-terminated compound is a hydroxyl-terminated linear (C₁-C₆)alkyl disulfide. In still further embodiments, the hydroxyl-terminated compound is a hydroxyl-terminated linear (C₁-C₄)alkyl disulfide. In some exemplary embodiments, the hydroxyl-terminated compound is 2-hydroxyethyl disulfide.

According to some embodiments, the diisocyanate compound is selected from the group consisting of isophorone diisocyanate (IPDI), 1,4-tetramethylene diisocyanate, 1,6-hexamethylene diisocyanate (HDI), and 1,10-decamethylene diisocyanate. Each possibility represents a separate embodiment. In some exemplary embodiments, the diisocyanate compound is IPDI.

According to some embodiments, the HTPB comprises 1,3-butadiene derived-monomer units of formula (B1), formula (B2), and formula (B3), as presented hereinabove.

In some embodiments, the proportion of the monomer unit of formula (B1) in the entirety of the 1,3-butadiene-derived monomer units present in the HTPB is 10 to 60 mole percent, including each value within the specified range. In further embodiments, the proportion of the monomer unit of formula (B1) in the entirety of the 1,3-butadiene-derived monomer units present in the HTPB is 10 to 30 mole percent, including each value within the specified range.

In some embodiments, the proportion of the monomer unit of formula (B2) in the entirety of the 1,3-butadiene-derived monomer units present in the HTPB is 20 to 70 mole percent, including each value within the specified range. In further embodiments, the proportion of the monomer unit of formula (B2) in the entirety of the 1,3-butadiene-derived monomer units present in the HTPB is 50 to 70 mole percent, including each value within the specified range.

In some embodiments, the proportion of the monomer unit of formula (B3) in the entirety of the 1,3-butadiene-derived monomer units present in the HTPB is 10 to 50 mole percent, including each value within the specified range. In further embodiments, the proportion of the monomer unit of formula (B3) in the entirety of the 1,3-butadiene-derived monomer units present in the HTPB is 10 to 30 mole percent, including each value within the specified range.

In certain embodiments, the HTPB comprises about 20 mole percent monomer units of formula (B1), 60 mole percent monomer units of formula (B2), and 20 mole percent monomer units of formula (B3). According to additional embodiments, the HTPB comprises from about 0 to about 80 mole percent of 1,3-butadiene derived-monomer units in which the C═C double bond is hydrogenated. In further embodiments, the HTPB comprises from about 10 to about 70 mole percent of 1,3-butadiene derived-monomer units in which the C═C double bond is hydrogenated. In still further embodiments, the HTPB comprises from about 20 to about 60 mole percent of 1,3-butadiene derived-monomer units in which the C═C double bond is hydrogenated. In yet further embodiments, the HTPB comprises from about 30 to about 50 mole percent of 1,3-butadiene derived-monomer units in which the C═C double bond is hydrogenated.

In some embodiments, the HTPB has a formula (C1):

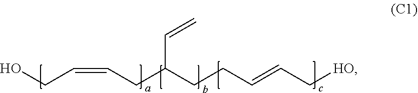

wherein a ranges from about 0.1 to about 0.5, b ranges from about 0.1 to about 0.6, and c ranges from about 0.2 to about 0.7, including each value within the specified ranges. In further embodiments, the hydroxyl-terminated polybutadiene polymer has a formula (C1), wherein a ranges from about 0.1 to about 0.3, b ranges from about 0.1 to about 0.36, and c ranges from about 0.5 to 0.7, including each value within the specified ranges.

In some exemplary embodiments, the HTPB has a formula (C2):

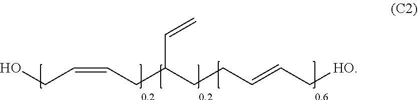

According to some embodiments, the HTPB has a molecular weight ranging from about 1,000 g/mole to about 6,000 g/mole, including each value within the specified range. According to further embodiments, the HTPB has a molecular weight ranging from about 2,000 g/mole to about 5,000 g/mole, including each value within the specified range.

The molar ratio between the HTPB and the hydroxyl-terminated compound can range from about 1:1 to about 1:3, including all iterations of ratios within the specified range. In some embodiments, the ratio between the HTPB and the hydroxyl-terminated compound is 1:1. The molar ratio between the HTPB and the diisocyanate compound can range from about 1:2.1 to about 1:4.2, including all iterations of ratios within the specified range. In some embodiments, the molar ratio between the HTPB and the diisocyanate compound is 1:2.1. In certain embodiments, the molar ratio between the HTPB, the hydroxyl-terminated compound, and the diisocyanate compound is about 1:1:2.1.

According to some embodiments, a reaction between the hydroxyl-terminated polybutadiene (HTPB), the diisocyanate compound and the hydroxyl-terminated compound is catalyzed by a catalyst selected from the group consisting of dibutyltin dilaurate (DBTDL), dibutyltin diacetate, dibutyltin mercaptide, dibutyltin dilauryl mercaptide, cobalt bis(2-ethyl hexanoate), bismuth tris(2-ethyl hexanoate), tertiary amine, and any combination thereof. Each possibility represents a separate embodiment. In certain embodiments, said catalyst is DBTDL.

According to some embodiments, the HTPB is first mixed with the hydroxyl-terminated compound to form a homogenous mixture. In further embodiments, the isocyanate compound and the catalyst are added dropwise to the homogeneous mixture.

According to some embodiments, the reaction is performed at a temperature ranging from −30 to 200° C., including each value within the specified range. In further embodiments, the reaction is performed at a temperature ranging from 0 to 175° C., including each value within the specified range. In yet further embodiments, the reaction is performed at a temperature ranging from 20 to 150° C., including each value within the specified range. In yet further embodiments, the reaction is performed at a temperature ranging from 40 to 120° C., including each value within the specified range. In still further embodiments, the reaction is performed at a temperature ranging from 60 to 100° C., including each value within the specified range. In some exemplary embodiments, the reaction is performed at a temperature of about 80° C.

According to some embodiments, the reaction is performed for up to about 96 hours. According to further embodiments, the reaction is performed for up to about 72 hours. In yet further embodiments, the reaction is performed for up to about 48 hours. In still further embodiments, the reaction is performed for up to about 24 hours. In yet further embodiments, the reaction is performed for up to about 16 hours. In still further embodiments, the reaction is performed for up to about 12 hours. In yet further embodiments, the reaction is performed for up to about 9 hours. In still further embodiments, the reaction is performed for up to about 8 hours.

According to some embodiments, the reaction is performed for at least about 6 hours. According to further embodiments, the reaction is performed for at least about 8 hours.

According to some embodiments, the reaction product is subjected to at least one dissolution-precipitation-decantation procedure. In some embodiments, the dissolution-precipitation-decantation procedure is performed three times. The dissolution-precipitation-decantation procedure can include a step of dissolving the mixture in a first solvent to form a homogeneous solution. In some embodiments, the dissolution-precipitation-decantation procedure further comprises a step of adding a second solvent to the solution to induce precipitation of a product. In some embodiments, the dissolution-precipitation-decantation procedure further comprises a step of separating the product from the solution. The first solvent can be a nonpolar organic solvent and the second solvent can be a polar organic solvent. In further embodiments, the product obtained following the at least one dissolution-precipitation-decantation procedure is dissolved in the first solvent and cast into a predefined mold to obtain an elastomer film following evaporation of the first solvent.

According to some embodiments, the reaction mixture comprises, in addition to the reactants and the catalyst, at least one component selected from the group consisting of plasticizers, pigments, organic or inorganic fillers, adhesion promoter, UV-stabilizers, rheology modifiers, and flame-retardant additives. Each possibility represents a separate embodiment. Solvents, plasticizers, pigments, organic or inorganic fillers, adhesion promoter, UV-stabilizers, rheology modifiers, flame-retardant additives, are those used in polymer manufacturing and are well-known for those skilled in the art. Reference is made, for instance, to Harper, “Modern Plastics Handbook”, Chapter 4, 1999, pages 4.1-5.0; Wypych, “Handbook of Plasticizers”, Ed.: ChemTec Publishing, Chapter 11, 2004, pages 273-379; and Bolgar et al. “Handbook for the chemical analysis of plastics and polymer additives”, Ed.: CRC Press, Chapters 3 to 9, 2008, pages 27-303.

In another aspect, there is provided an antibacterial composition comprising the elastomer according to the various aspects and embodiments hereinabove, and a quaternary ammonium compound (QAC). Non-limiting examples of quaternary ammonium compounds within the scope of the present invention include cetyltrimethylammonium bromide (CTAB), lauroyl trimethyl ammonium bromide (LTAB), myristyl trimethyl ammonium chloride (MTAC), cetyl trimethyl ammonium chloride (CTAC), cetrimide, stearoyl trimethyl ammonium chloride (STAC), stearoyl trimethyl ammonium bromide (STAB), benzalkonium chloride (alkyldimethylbenzylammonium chloride), N-cetylpyridinium bromide (N-hexadecylpyridinium bromide), N-cetylpyridinium chloride (N-hexadecylpyridinium chloride), benzyl dimethyl tetradecyl ammonium chloride, and benzyl dimethyl hexadecyl ammonium chloride. Each possibility represents a separate embodiment.

In some exemplary embodiments, the QAC is CTAB.

In some embodiments, the QAC is present in the antibacterial composition in a weight percent of up to about 3% of the total weight of the composition. In certain embodiments, the QAC is present in the antibacterial composition in a weight percent of up to about 1% of the total weight of the composition.

According to some embodiments, the antibacterial composition is in a form of an antibacterial elastomer film.

The antibacterial composition can be prepared by dissolving the elastomer according to the various aspects and embodiments hereinabove in a suitable solvent, such as, for example the first solvent, and mixing it with the dissolved QAC to form a homogeneous mixture. The antibacterial elastomer film can be prepared by casting the homogeneous mixture into a predefined mold and evaporating the solvent.

According to some embodiments, the antibacterial elastomer film has a tensile strength value above about 5 MPa at room temperature. The terms “tensile strength” and “ultimate tensile stress”, as used herein interchangeably, refer to the maximum stress that a material can withstand while being stretched or pulled before failing or breaking.

According to further embodiments, the tensile stress of the antibacterial elastomer film is above about 7.5 MPa at room temperature. According to yet further embodiments, the tensile stress of the antibacterial elastomer film is above about 10 MPa at room temperature.

According to some embodiments, the antibacterial elastomer film has an elongation at break value higher than 500% at room-temperature. The term “elongation at break”, as used herein, refers to the maximum elongation that a material can withstand while being stretched or pulled before failing or breaking. According to further embodiments, the antibacterial elastomer film has an elongation at break value higher than 750% at room-temperature. According to yet further embodiments, the antibacterial elastomer film has an elongation at break value higher than 1000% at room-temperature.

The term “room-temperature” denotes a temperature ranging from 15 to 30° C., including each value within the specified range.

According to some embodiments, the antibacterial elastomer film has a self-healing efficiency of at least about 50% at room temperature.

The term “self-healing efficiency”, as used herein, refers in some embodiments to the ability of the antibacterial elastomer film to retain its original tensile stress and/or elongation at break parameters following mechanical damage to a certain extent. For example, in the context of the present invention, a self-healing efficiency of about 50% can refer to the ability of the antibacterial elastomer film to retain 50% of its original tensile stress and/or elongation at break parameters. The term “original”, as used in connection to the mechanical parameters, refers to these parameters before the infliction of the mechanical damage.

According to some embodiments, the antibacterial elastomer film has a self-healing efficiency of at least about 60% at room temperature. According to some embodiments, the antibacterial elastomer film has a self-healing efficiency of at least about 70% at room temperature.

In some embodiments, the antibacterial elastomer film retains at least about 50% of its original tensile strength at room temperature following mechanical damage. In some embodiments, the antibacterial elastomer film retains at least about 50% of its original elongation at break at room temperature following mechanical damage.

In another aspect, there is provided a wound dressing comprising a film made of the elastomer (i.e., the “elastomer film”), or of the antibacterial composition (i.e., the “antibacterial elastomer film”), according to the various aspects and embodiments hereinabove. Said wound dressing can be used to assist in the healing of a wound by placing said dressing over the wound. Additionally, said would dressing can be used to assist in a surgical incision of a body part, wherein the wound dressing is applied to said body part and the incision is performed atop the wound dressing.

The term “wound dressing”, as used herein, refers to a dressing for topical application to a wound.

The term “wound”, as used herein, refers to an injury to any tissue, including intentional injuries, such as for example, surgical incision and unintentional injuries, including, inter alia, acute wounds, delayed or difficult to heal wounds, and chronic wounds. Each possibility represents a separate embodiment. Examples of wounds may include both open and closed wounds. The term “wound” may also include for example, injuries to the skin and subcutaneous tissue initiated in different ways (e.g., incisions, wounds induced by trauma or abrasion, and pressure sores) and with varying characteristics. Wounds may be classified into one of four grades depending on the depth of the wound: i) Grade I wounds limited to the epithelium; ii) Grade II wounds extending into the dermis; iii) Grade III wounds extending into the subcutaneous tissue; and iv) Grade IV wounds (or full-thickness wounds) wherein bones are exposed (e.g., a bony pressure point such as the greater trochanter or the sacrum). In certain embodiments, the wound dressing is intended for the treatment of an open wound. In additional embodiments, the wound is selected from a Grade II, a Grade III, and a Grade IV wound with each possibility representing a separate embodiment.

The wound dressing can further be a multifunctional wound dressing (also termed herein “smart wound dressing”). In certain such embodiments, the wound dressing further comprises at least one sensor.

According to some embodiments, the elastomer film or the antibacterial elastomer film constitutes a substrate of the sensor, thereby supporting other electronic components of the device, while imparting biocompatibility and self-healing ability thereto.

The sensor can be configured as any one of the various types of electronic devices, including, but not limited to, resistive sensors, chemiresistive sensors, capacitive sensors, impedance sensors, field effect transistor sensors, strain gauge sensors and the like, or combinations thereof. Each possibility represents a separate embodiment.

The substrate can have any desirable geometry. In rectangular geometries, the length and/or width of the substrate can range between about 0.01-100 mm, including each value within the specified range. The thickness of the substrate can range between about 0.1-10 mm, including each value within the specified range.

The sensor can be selected from the group consisting of a chemical sensor, an electrochemical sensor, a biological sensor, and a physical sensor. Each possibility represents a separate embodiment. The term “chemical sensor”, as used herein, refers to a device comprising a chemical entity, which detects the presence of an analyte. The chemical sensor can comprise a sensor element whose properties, such as, but not limited to, physical, optical or morphological properties are modified in the presence of an analyte. The term “electrochemical sensor”, as used herein, refers to a device which is adapted for performing at least one electrochemical measurement to detect the presence of an analyte. The electrochemical sensor can be configured in a form selected, inter alia, from a resistive sensor, a capacitive sensor, a chemiresistive sensor, and an impedance sensor. Each possibility represents a separate embodiment. A non-limiting example of an electrochemical sensor is a pH sensor based on a protonated electrically conducting polymer. The term “biological sensor”, as used herein, refers to a device comprising a biological component, which detects the presence of an analyte in a biological sample. A non-limiting example of a biological sensor is a glucose sensor. The term “physical sensor”, as used herein, refers to a device which senses the absolute value or a change in a physical quantity and generates a corresponding signal or data. Examples of a physical quantity include, but are not limited to, temperature, pressure, humidity, level precipitation, flow rate, pH, coefficient of friction, intensity of light, intensity of sound, intensity of radio waves, and the like. Each possibility represents a separate embodiment.

According to some embodiments, the at least one sensor is configured to detect one or more parameters of the wound. The term “parameter of the wound”, as used herein, refers to a parameter associated with a physiological and/or chemical environment of the wound, which may change as a result of the wound healing process. Non-limiting examples of such parameters, which detection can assist in the monitoring and evaluation of the wound healing process, include glucose concentration, uric acid concentration, pH, temperature, and humidity. Each possibility represents a separate embodiment. Accordingly, in some embodiments, the at least one sensor is selected from the group consisting of a glucose sensor, a pH sensor, a temperature sensor, a uric acid sensor, a humidity sensor, a volatile organic compounds sensor (VOCs), an impedance sensor, and a pressure sensor. Each possibility represents a separate embodiment.

According to some embodiments, the at least one sensor is embedded within or deposited onto the substrate. In further embodiments, the at least one sensor comprises an electrode and a sensing layer disposed on a portion of said electrode and/or electrically connected thereto. In some embodiments, the at least one sensor further comprises a reference electrode.

In some embodiments, the wound dressing comprises a plurality of sensors. In further embodiments, the wound dressing comprises a plurality of electrodes.

The electrodes can comprise any metal having high conductivity. The electrode and/or the reference electrode can be made of a micro-sized or nanosized conductive material.

The term “micro-sized”, as used herein, refers to material having a mean particle size in the range of above 1 μm but below 1,000 μm, including each value within the specified range.

The term “nanosized”, as used herein, refers to material having a mean particle size in the range of above 0.5 nm but below 1,000 nm, including each value within the specified range.

The term “particle size”, as used herein, refers to the length of the particle of the material in the longest dimension thereof.

Said conductive material can be embedded within the substrate. In additional embodiments, the conductive material is deposited onto the substrate. Non-limiting examples of conductive materials suitable for use in the sensors according to the principles of the present invention include metals, metal alloys, metal carbides, metal nitrides, metal oxides, metal silicides, carbon, polymers, ceramics, and combinations thereof. Each possibility represents a separate embodiment. According to certain embodiments, the conductive material is a nanosized material having a form selected from the group consisting of nanoparticles, nanowires, nanotubes, nanoflakes, nanofibers, nanoribbons, nano-whiskers, nanostrips, nanorods, and combinations thereof. Each possibility represents a separate embodiment. In some exemplary embodiments, the conductive material is selected from nanowires, nanotubes and combinations thereof. Each possibility represents a separate embodiment. In certain embodiments, said nanowires are silver nanowires. In additional embodiments, said nanotubes are carbon nanotubes.

The electrodes can have any suitable shape, as known in the art. In certain embodiments, the electrodes have an elongated shape. In further embodiments, the electrodes are arranged on the substrate with their longest dimension being parallel to the longitudinal axis of the substrate. In some embodiments, the electrodes are disposed on one part of the substrate, wherein their respective sensing layers are disposed essentially in the center of the substrate. This way, the sensors can continue monitoring the wound condition, wherein the wound dressing is applied with its center to the wound, even if the wound dressing is damaged proximally to the wound. Furthermore, if the wound dressing is being used in a surgical incision and is cut in half, the specific position of the electrodes and the sensing layer allows to prevent damage to the sensors, so that the sensors can monitor the intended incision area before the surgery and monitor the wound following the surgery.

According to some embodiments, the sensing layer comprises a material selected from the group consisting of a biorecognition element, a redox-active element, an electrically conducting material, an ion-conducting material, a thermally conductive material, and any combination thereof. Each possibility represents a separate embodiment.

The term “biorecognition element”, as used herein, refers to a compound, which is selective to a constituent or biomarker present within the wound, such as, e.g., serum or interstitial fluid. Non-limiting examples of biorecognition elements include an enzyme, an antibody, an aptamer, an ion-selective membrane (ISM), a protonically doped polymer, DNA, ABTS (2,2′-azino-bis(3-ethylbenzothiazoline-6-sulfonic acid)), molecularly imprinted polymer (MIP), and combinations thereof. Each possibility represents a separate embodiment. In certain embodiments, the biorecognition element is glucose oxidase or glucose dehydrogenase.

Non-limiting examples of electrically conducting materials suitable for use in the sensing layer of the wound dressing of the present invention include metal nanoparticles, metal nanowires, graphene, carbon nanotubes (CNTs), and polymers (such as polyaniline (PANI) and poly(3,4-ethylenedioxythiophene)-poly(styrenesulfonate), (PEDOT:PSS)). Each possibility represents a separate embodiment. The electrically conducting materials, such as, for example, metal nanoparticles or CNTs can by capped with an organic layer.

In some embodiments, the thermally conductive material is characterized by a temperature-dependent electrical resistance. A non-limiting example of thermally conductive material is reduced graphene oxide.

The term “redox-active element”, as used herein, refers to a molecule or component of a molecule that is capable of being oxidized or reduced under the conditions of use.

The sensing layer can further include additives selected from, but not limited to, an adhesive material, a hydrogen peroxide transducer, and an immobilizing layer. Each possibility represents a separate embodiment.

In some embodiments, the sensing layer comprises a material selected from the group consisting of polyethyleneimine (PEI), glucose oxidase (GOx), carbon nanotubes, reduced graphene oxide (rGO), polyaniline (PANI), K₃[Fe(CN)₆] (Prussian blue), and any combination thereof. Each possibility represents a separate embodiment.

In some exemplary embodiments, the wound dressing comprises: a glucose sensor comprising an electrode made of Ag nanowires and a sensing layer comprising Prussian blue and glucose oxidase; a pH sensor comprising an electrode made of Ag nanowires and a sensing layer comprising PANI; and a temperature sensor comprising an electrode made of Ag nanowires and a sensing layer comprising PEI and reduced graphene oxide.

According to some embodiments, the wound dressing further comprises at least one reference electrode made of Ag nanowires, which are partially coated with an Ag/AgCl paste.

The wound dressing can further comprise an additional film made of the elastomer or the antibacterial composition according to the various aspects and embodiments hereinabove, wherein the additional film covers at least a portion of the sensor. In some embodiments, the additional film covers the electrode but does not cover the sensing layer. In certain embodiments, at least a portion of the sensing layer is not covered by the additional film.

According to some embodiments, the substrate has a thickness ranging from about 500 μm to about 5 mm, including each value within the specified range.

According to some embodiments, the additional film has a thickness ranging from about 100 μm to about 2 mm, including each value within the specified range.

According to some embodiments, the wound dressing further comprises at least one of a drug release layer, self-cleaning protecting layer, and wearable data processing device. Each possibility represents a separate embodiment.

The wound dressing according to the various aspects and embodiments hereinabove can be for use in the treatment and/or monitoring a condition of a wound.

In some embodiments, the condition of the wound is monitored by the at least one sensor.

In another aspect, there is provided a method of treating and/or monitoring a condition of a wound, comprising applying the wound dressing according to the various aspects and embodiments hereinabove to the wound.

In some embodiments, the method further comprises measuring one or more parameters of the wound by the at least one sensor.

In another aspect, there is provided a method of performing a surgical incision on a body part of a subject, the method comprising: applying the wound dressing according to the various aspects and embodiments hereinabove to said body part; performing the incision atop the wound dressing, thereby separating the elastomer film of the wound dressing into at least two segments; and connecting said at least two segments.

In some embodiments, the method further comprises measuring one or more parameters of said body part by the at least one sensor. In further embodiments, said measurement is performed prior to the step of performing the incision and/or following the step of connecting said at least two segments.

According to some embodiments, the wound dressing is for use in performing a surgical incision on a body part, wherein the wound dressing is applied to said body part and the incision is performed atop the wound dressing.

As used herein and in the appended claims the singular forms “a”, “an,” and “the” include plural references unless the context clearly dictates otherwise. Thus, for example, reference to “a polymeric chain” can include a plurality of such polymeric chains and equivalents thereof known to those skilled in the art, and so forth. It should be noted that the term “and” or the term “or” is generally employed in its sense including “and/or” unless the context clearly dictates otherwise.

As used herein, the term “about”, when referring to a measurable value such as an amount, a temporal duration, and the like, is meant to encompass variations of ±10%, more preferably ±5%, even more preferably ±1%, and still more preferably ±0.5% from the specified value, as such variations are appropriate to perform the disclosed methods.

The following examples are presented in order to illustrate some embodiments of the invention more fully. They should, in no way be construed, however, as limiting the broad scope of the invention. One skilled in the art can readily devise many variations and modifications of the principles disclosed herein without departing from the scope of the invention.

### EXAMPLES

### Example 1—Preparation of the Elastomer Comprising 1,10-Decanediol as a Chain Extender (PUIDE)

HTPB (2.1 g, 1 mmol) was first heated at 80° C. under vacuum for 2 h to remove any moisture. Then, 1,10-decanediol (DE, 174 mg, 1 mmol) as a chain extender was added to the HTPB under N₂ atmosphere. Following the formation of a homogenous viscous liquid, IPDI (467 mg, 2.1 mmol) and DBTDL (5 mg, ˜1,600 ppm) were added dropwise into the vessel and stirred until the magnet could no longer rotate.

Subsequently, the mixture was placed into the oven (80° C.) for 8 h. The mixture was then dissolved in chloroform to form a homogeneous solution. Then, MeOH (30 mL) was added to afford precipitation of the product. White precipitate-like viscous liquid appeared and the mixture was settled for 30 minutes. Then, the upper solution was decanted. 15 mL chloroform was then added to dissolve the product. The dissolution-precipitation-decantation was repeated three times and the final product (PUIDE) solution was poured into a rectangle Teflon mold and allowed to slowly evaporate at room temperature overnight.

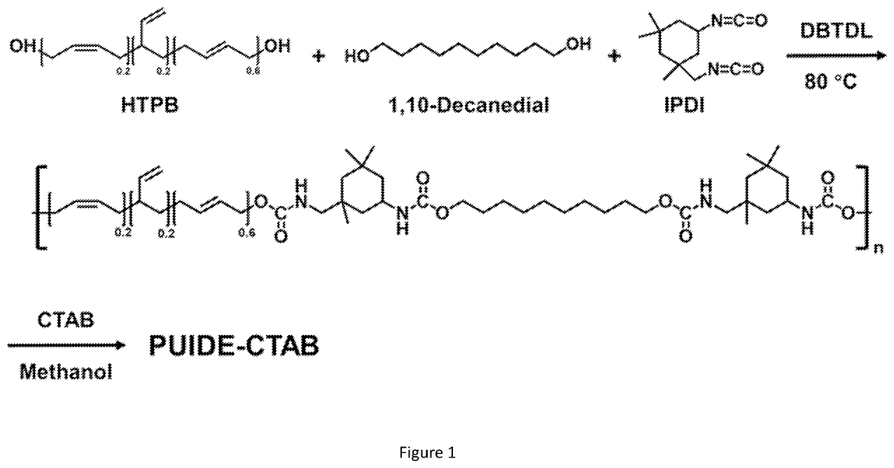

The polymerization reaction and the chemical structure of the obtained PUIDE elastomer are shown in FIG. 1.

### Example 2—Preparation of the Antibacterial Composition Comprising the Elastomer Comprising 1,10-Decanediol as a Chain Extender and CTAB (PUIDE-CTAB)

For synthesizing PUIDE-CTAB, CTAB (dissolved in MeOH) with a mass ratio of 1% to PUIDE was added to the PUIDE solution, and the mixture was stirred to form a homogeneous solution using vortex oscillator. Then, the final mixed solution was poured into a Teflon mold and allowed to slowly evaporate at room temperature overnight. Thereafter, the resulting film was dried in a vacuum oven at 80° C. for 24 hours to remove residual solvent, resulting in a light yellow transparent film of PUIDE-CTAB.

### Example 3—Preparation of the Elastomer Comprising 2-Hydroxyethyl Disulfide as a Chain Extender (PIUDS)

HTPB (2.1 g, 1 mmol) was first heated at 80° C. under vacuum for 2 h to remove any moisture. Then, 2-hydroxyethyl disulfide (HEDS) as a chain extender was added to HTPB under N₂ atmosphere. Following the formation of a homogenous viscous liquid, IPDI (467 mg, 2.1 mmol) and DBTDL (5 mg, ˜1,600 ppm) were added dropwise into the vessel and stirred until the magnet could no longer rotate. Subsequently, the mixture was placed into the oven (80° C.) for 8 h. The mixture was then dissolved in chloroform to form a homogeneous solution. Then, MeOH (30 mL) was added to afford precipitation of the product. White precipitate-like viscous liquid appeared and the mixture was settled for 30 minutes. Then, the upper solution was decanted. 15 mL chloroform was then added to dissolve the product. The dissolution-precipitation-decantation was repeated three times and the final product (PUIDS) solution was poured into a rectangle Teflon mold and allowed to slowly evaporate at room temperature overnight.

The polymerization reaction and the chemical structure of the obtained PUIDE elastomer are shown in FIG. 1.

Various PUIDS elastomers were prepared with different HTPB:HEDS:IPDI molar ratios, as shown in Table 1. The obtained elastomers are characterized by different contents of the S—S residue within a repeating unit of the elastomer.

TABLE 1
Weights and molar ratios of the reactants
in the synthesis of PUIDS.

| Elastomer | HTPB | HEDS | IPDI | Molar ratio of | S—S |
|---|---|---|---|---|---|
| name | [g] | [mg] | [mg] | (HTPB:HEDS:IPDI) | content % |
| PUIDS-1 | 2.1 | 154 | 467 | 1:1:2.1 | 2.35 |
| PUIDS-2 | 2.1 | 308 | 689 | 1:2:3.1 | 4.13 |
| PUIDS-3 | 2.1 | 462 | 911 | 1:3:4.1 | 5.35 |

### Example 4—Preparation of the Antibacterial Composition Comprising the Elastomer Comprising 2-Hydroxyethyl Disulfide as a Chain Extender and CTAB (PUIDS-CTAB)

For synthesizing PUIDS-CTAB, CTAB (dissolved in MeOH) with a mass ratio of 1% to PUIDS was added to the PUIDE solution, and the mixture was stirred to form a homogeneous solution using vortex oscillator. Then, the final mixed solution was poured into a Teflon mold and allowed to slowly evaporate at room temperature overnight. Thereafter, the resulting film was dried in a vacuum oven at 80° C. for 24 hours to remove residual solvent, resulting in a light yellow transparent film of PUIDS-CTAB.

### Example 5—Physical Characterizations of PUIDE-CTAB and PUIDS-CTAB PUIDE-CTAB

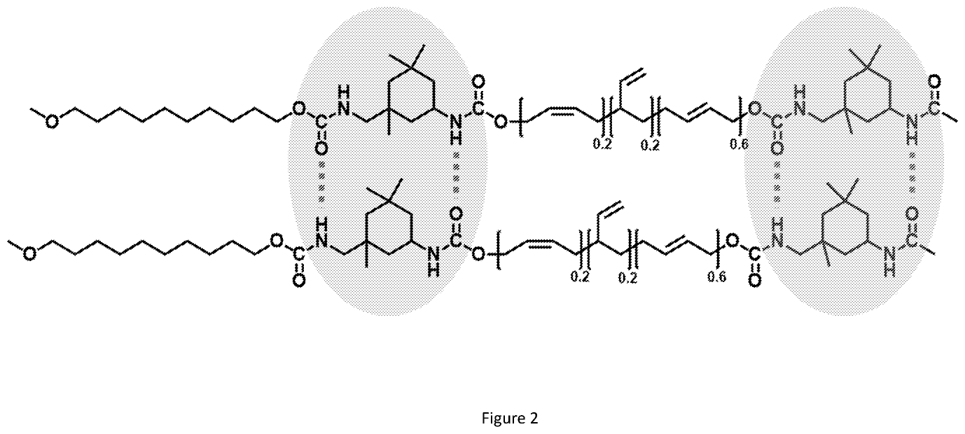

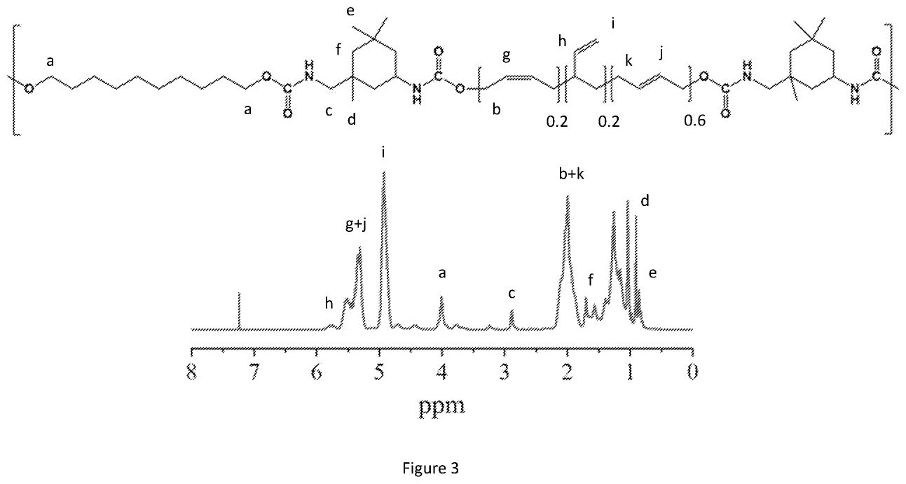

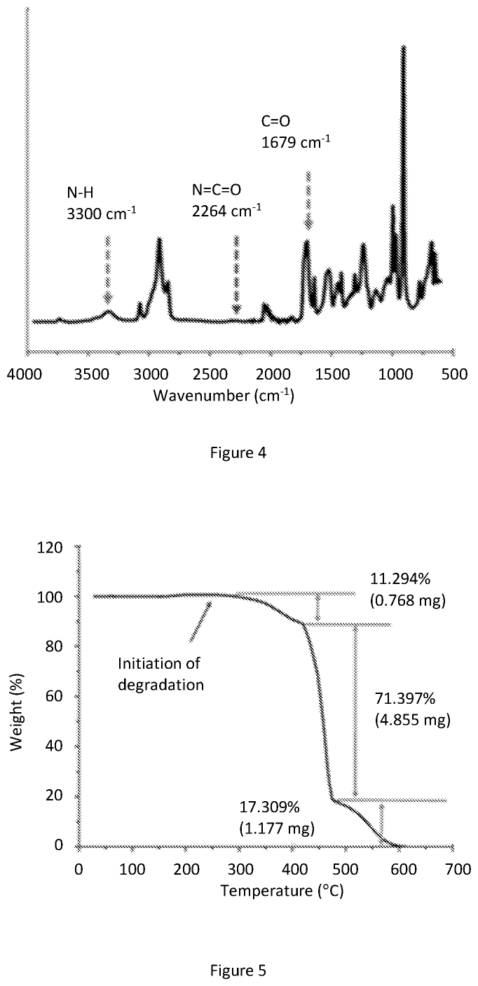

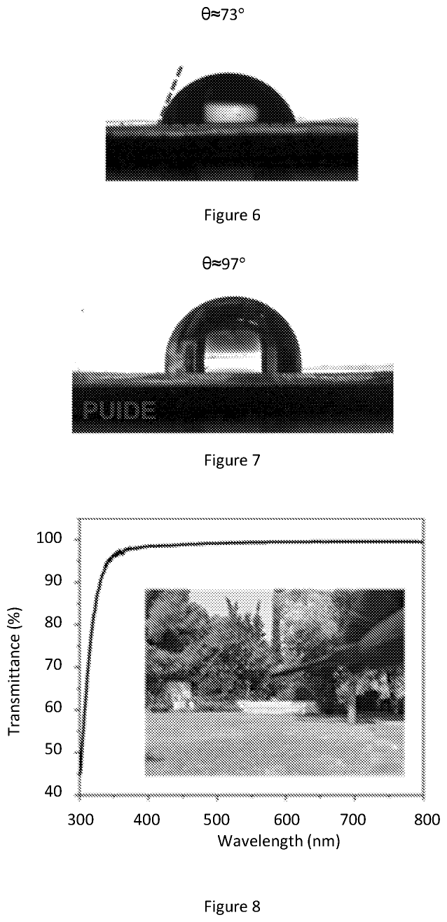

Without wishing to being bound by theory or mechanism of action, it is contemplated that the HTPB parts in the synthetic elastomer act as soft components, which contribute to favorable flexibility, while the DE and IPDI together are regarded as the hard segment by contributing hydrogen bonds in the formation of urea/urethane linkages between the elastomer chains (FIG. 2), playing a key role in the mechanical and self-healing properties of the elastomer. The ¹H-NMR spectrum of PUIDE-CTAB is shown in FIG. 3. Negligible peaks of the N═C═O stretching bond at 2,264 cm⁻¹ in the FTIR spectrum demonstrated that the diisocyanate monomers had been fully converted to urethane bonds, which is also the premise of forming many hydrogen bonds in the elastomer (FIG. 4). The PUIDE-CTAB showed great stability up to temperatures as high as 240° C. (FIG. 5). From contact angle measurements, it was noticed that PUIDE-CTAB films have a favorable hydrophilicity compared with PUIDE (FIGS. 6 and 7). FIG. 8 shows that the average transmittance of the PUIDE-CTAB film with a thickness of 200 m under visible light wavelengths (400-750 nm) was >97%. The inset optical photograph clearly depicts the high transparency of the film.

### PUIDS-CTAB

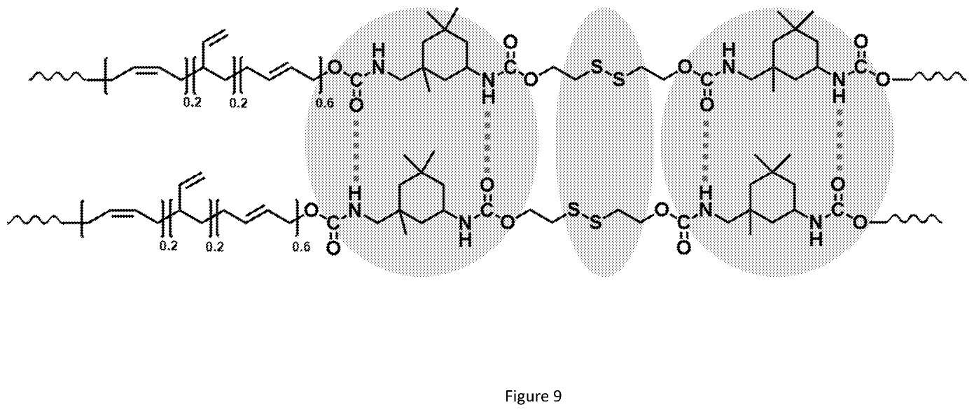

Without wishing to being bound by theory or mechanism of action, it is contemplated that the HTPB parts in the synthetic elastomer act as soft components, which contribute to favorable flexibility, while IPDI is regarded as the hard segment by contributing hydrogen bonds in the formation of urea/urethane linkages (FIG. 9) and HEDS contributes to the formation of dynamic disulfide linkages between the elastomer chains, playing a key role in the mechanical and self-healing properties of the elastomer.

### Example 6—Mechanical and Self-Healing Characterizations of PUIDE-CTAB and PUIDS-CTAB

Mechanical tests were carried out by a universal Instron. The specimens were cut into a small dumbbell shape with a thickness of approximately 0.45 to 0.55 mm. The stretching rate was 100 mm/min unless stated otherwise.

Scratch self-healing processes were monitored using an optical microscope (BX51M, Olympus) equipped with a camera (LC20, Olympus).

Complete fracture self-healing measurements were done by cutting the specimens in half in air, and then two pieces of samples were manually merged and put into according positions. During the self-healing process, no external stress was applied to the interface. For underwater experiment, the specimens were immersed in water and cut in half, and then left to heal under the same conditions.

### PUIDE-CTAB

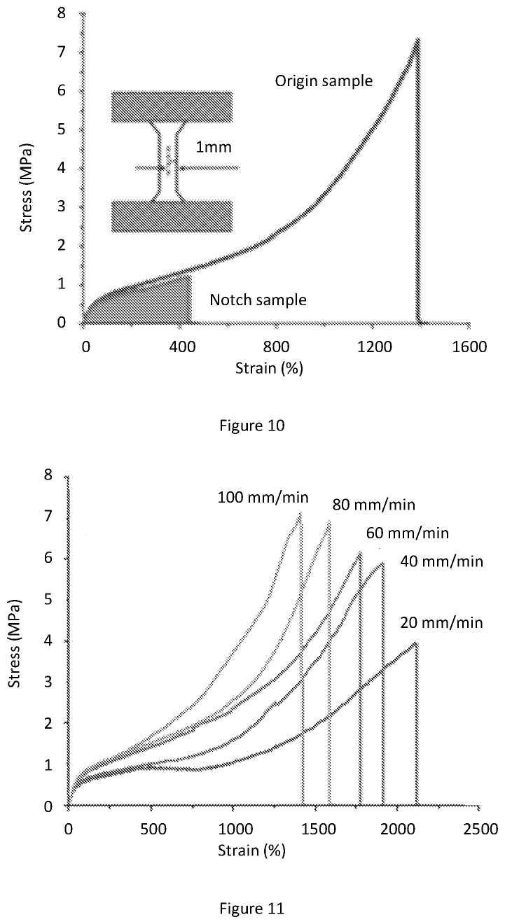

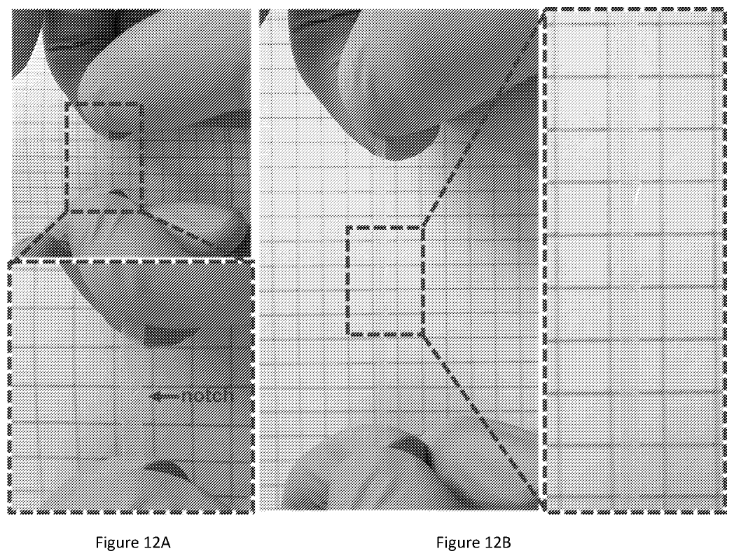

Excellent mechanical and self-healing properties are the key to achieving sutureless wound closure. Without wishing to being bound by theory or mechanism of action, it is contemplated that for PUIDE-CTAB, the hydrogen bonds in urea/urethane linkages serve as physical crosslinking points during the tensile process, maintaining the mechanically robust and stretchable polymer network. Broken hydrogen bonds can be gradually reconstructed after the release of external force. In order to evaluate the mechanical properties of PUIDE-CTAB samples, dumbbell specimens were subjected to a tensile test with a deformation rate of 100 mm/min. FIG. 10 shows that pristine PUIDE-CTAB has an ultimate tensile strength of 7.34 MPa and a favorable tensile strain of ˜1,400%. In particular, this elastomer showed an outstanding toughness of 35.98 MJ m⁻³, which is higher than previously reported values (Kim et al. Advanced Materials 30 (2018): 1705145; and Ying et al. ACS Applied Materials and Interfaces 12 (2020): 11072). The Young's modulus of PUIDE-CTAB was calculated at 1.45 MPa from its low-strain region, and the yield point of this elastomer appeared at a strain of 79.5%, indicating the initiation of dissociation of non-covalent hydrogen bonds (Filippidi et al. Science 358 (2017): 502). When the deformation rate was slower, the sample had higher stretchability (FIG. 11). To quantify the tear resistance property of the elastomer, tensile tests were run on a pre-damaged specimen with a 1 mm notch on the side (the size of the notch was half the width of the pristine specimen). During the elongation, the notch was blunted and could bear the strain up to 440% (FIG. 10 and FIGS. 12A-12B). The fracture energy, calculated using the Greensmith method, was 11.8 kJ m⁻², suggesting favorable notch-insensitivity of this elastomer (Wu et al. Advanced Materials 29 (2017): 1702616).

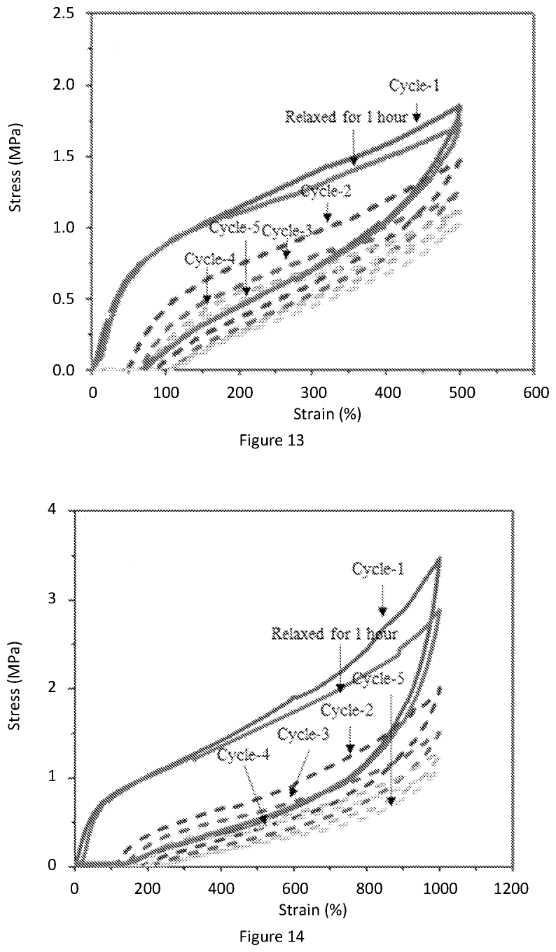

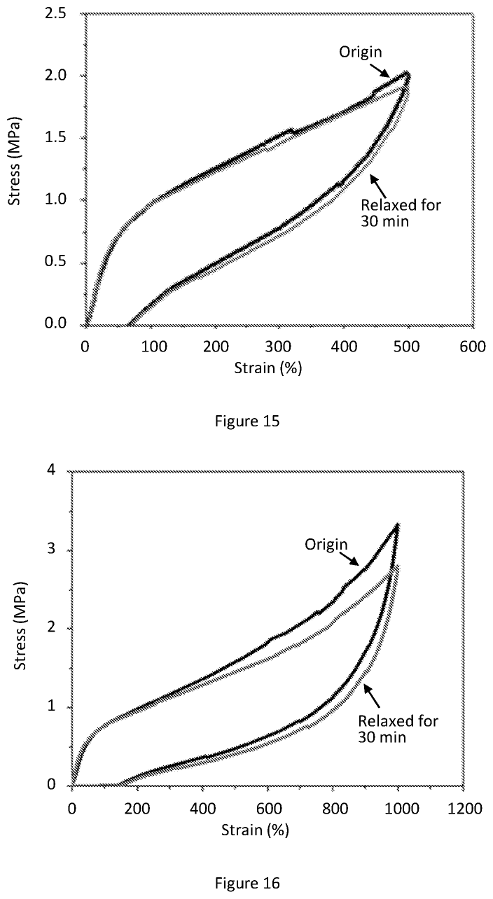

The elastomer had an obvious hysteresis loop in the first cycle, indicating significant energy dissipation (FIGS. 13 and 14). However, the hysteresis loop area in the second cycle was significantly reduced because the ruptured sacrificial bonds in first cycle did not have enough time to be reconstructed to their original state. This downward trend slightly decreased in the sequential cycles, implying continuous reorganization of the sacrificial bonds (Zhang et al. Advanced Materials 31 (2019): 1901402). After being at rest at room temperature for 1 h, the sample showed a loading/unloading curve similar to the original one, indicating the good fatigue resistance of the PUIDE-CTAB. With a 30 min rest after the first cycle test, the tensile strength and hysteresis loop area of the sample could be restored to the initial level, which indicates the reformation of the ruptured bonds and suggests the excellent self-recovery capability of PUIDE-CTAB (FIGS. 15 and 16).

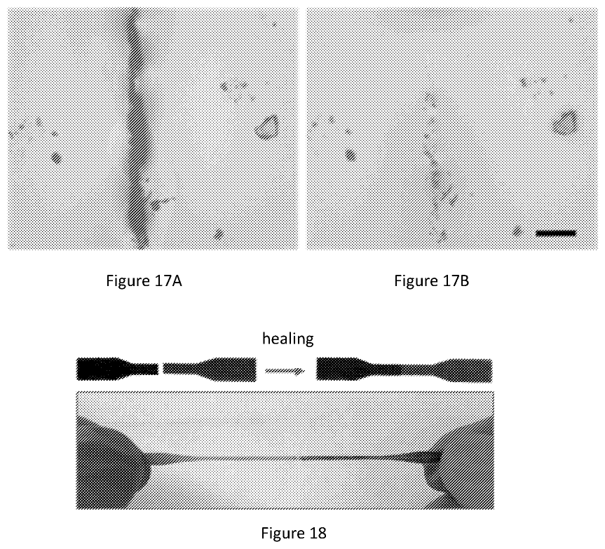

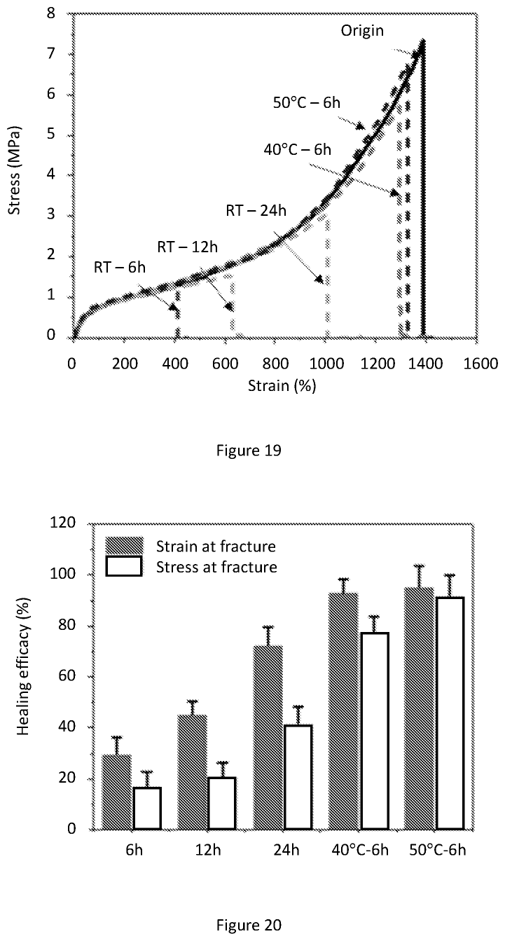

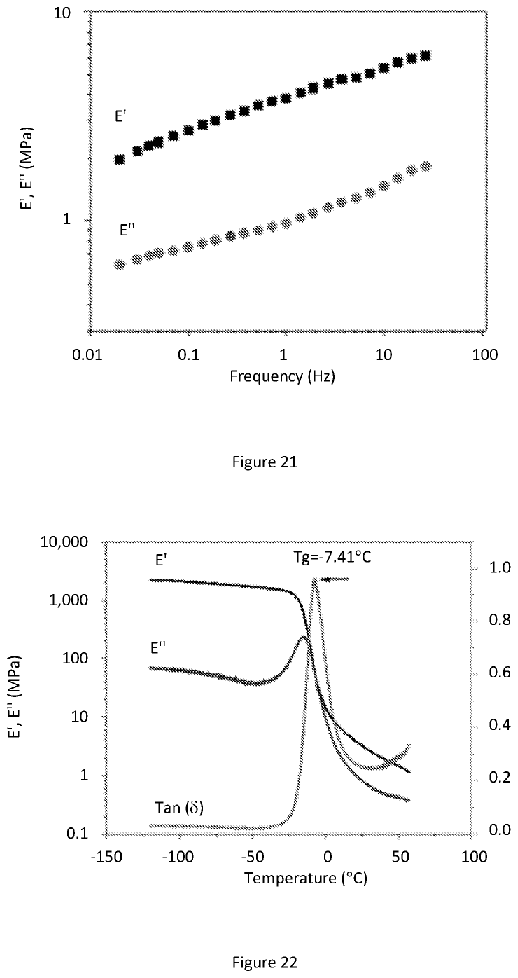

The artificial scar made on the PUIDE-CTAB surface almost vanished within 2 h at 40° C., demonstrating its excellent surface regeneration ability (FIGS. 17A-17B). Thereafter, the PUIDE-CTAB film was cut into 2 separate pieces, and the surfaces of the two half-films were gently pushed together at room temperature. Notably, the healed film could still withstand a large stretching deformation (FIG. 18). Similar behavior has been observed for complete cuts in underwater conditions. Subsequently, self-healing of PUIDE-CTAB was investigated (FIG. 19) in terms of its mechanical properties. The ultimate tensile strength and elongation at break point increased with heating and reached ˜95% of the pristine values after 6 h of self-healing at 50° C. (FIG. 20). The dynamic mechanical analysis (DMA) of PUIDE-CTAB indicated that the storage modulus (E′) was higher than the loss modulus (E″) at room temperature (FIG. 21), which implies that this elastomer behaves as an elastic solid at room temperature. As the temperature increases, both E′ and E″ decrease, indicating that the PUIDE-CTAB becomes more viscous at higher temperatures (FIG. 22). However, E′ is always higher than E″, demonstrating that the polymer maintains a good elastic solid form, which is of real significance in preventing deformation. These results imply that the PUIDE-CTAB has great mechanical and self-healing properties and can perform stably and reliably as dressing material in practical applications.

### PUIDS-1-CTAB

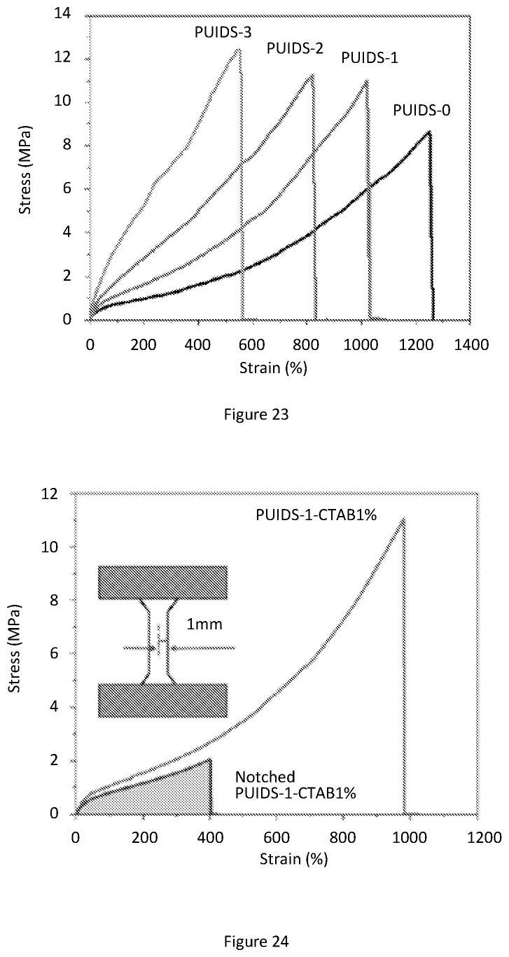

FIG. 23 shows typical Stress-Strain curves of the PUIDS elastomers, demonstrating high tensile stress for each of the PUID samples and simultaneous increase of the modulus and stiffness of the PUIDS polymers with the increase of disulfide content. Typical tensile Stress-Strain curves of original and notched PUIDS-1-CTAB samples are presented in FIG. 24. FIGS. 25A-25B present optical images of the scratched PUIDS-1-CTAB in the self-healing process at 60° C. The artificial scar made on the PUIDE-CTAB surface almost vanished within 2 h at 60° C., demonstrating its excellent surface regeneration ability.

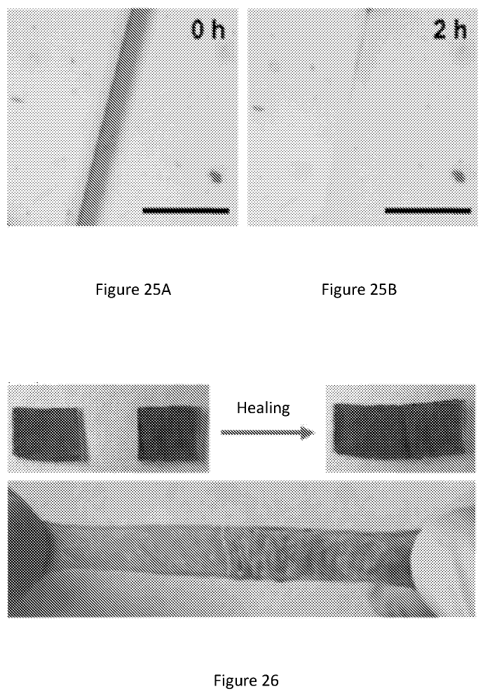

FIG. 26 shows photographs of the PUIDS-1-CTAB film after being cut into two separate pieces followed by a gentle push of these two half films together at room temperature. Notably, after healing for 30 min, the film was still able to withstand a large stretching deformation.

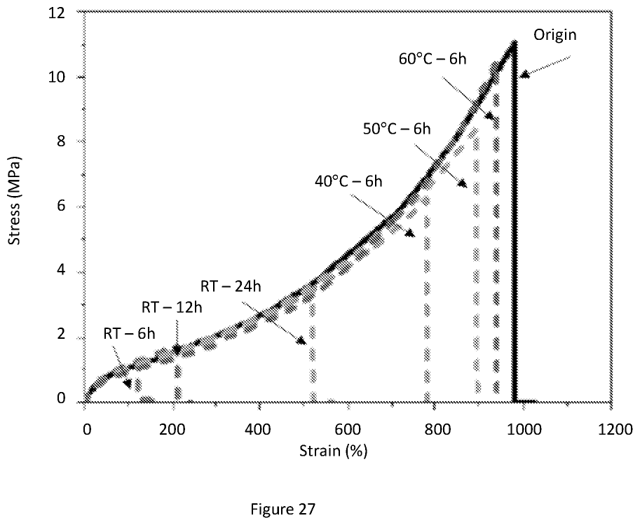

FIG. 27 shows Stress-Strain curves of original and full-cut PUIDS-1-CTAB samples healing at different temperatures for different periods of time.

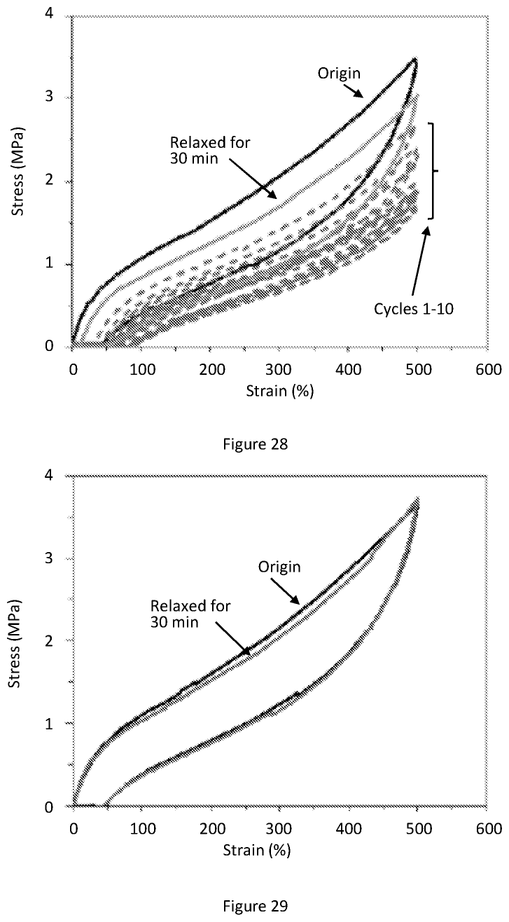

Fatigue resistance of PUIDS-1-CTAB as shown in FIG. 28 demonstrates an obvious hysteresis loop of the elastomer in the first cycle, indicating significant energy dissipation. However, the hysteresis loop area in the second cycle was significantly reduced because the ruptured sacrificial bonds in first cycle did not have enough time to be reconstructed to their original state. This downward trend slightly decreased in the sequential cycles, implying continuous reorganization of the sacrificial bonds. After being at rest at room temperature for 30 min, the sample showed a similar loading/unloading curve as the original one, indicating the good fatigue resistance of PUIDE-CTAB. FIG. 29 shows self-recovery of the loading/unloading curves of PUIDS-1-CTAB. As can be seen, with a 30 min rest after the first cycle test, the tensile strength and hysteresis loop area of the sample could be restored to the initial level, which indicates the reformation of the ruptured bonds and suggests the excellent self-recovery capability of PUIDS-1-CTAB.

### Example 7—In-Vitro and In-Vivo Biocompatibility of PUIDE-CTAB and PUIDS-CTAB

An ideal wound dressing should have good biocompatibility since it is in direct contact with blood and tissues. Therefore, to fully evaluate the biocompatibility of the elastomers, in-vitro (cytocompatibility and hemocompatibility) and in-vivo toxicity tests were performed.

### Cytotoxicity Evaluation

The cytocompatibility was assessed using normal human hepatic L02 cells by the cell count kit (CCK-8) and staining with Live/Dead kit. Briefly, human L02 cells were seeded into 96-well plates with 1640 culture medium and cultured for 24 h in an incubator (37° C., 5% CO₂). Then, the elastomer-CTAB samples were added into the 96-well plates. After additional 48 h of culture, 10 μL of CCK-8 was added and cultured in incubator for 1 h. The cell viability was measured at a wavelength of 450 nm by Biotek ELX800. Human L02 were stained with Live/Dead kit for 15 min in the dark and observed by Nikon ECLIPSE Ni.

### Hemolysis Assays

The hemolytic properties of the elastomer-CTAB samples were tested using mice blood (n=4). The mice blood was diluted in 2 mL of PBS buffer solution, followed by centrifugation at 2,000 rpm for 10 min to separate red blood cells (RBCs) from the serum. The precipitated RBCs were washed with PBS, and then diluted with 10 mL of PBS. Thereafter, 200 μL of the diluted RBCs suspension was mixed with 1 mL of PBS which contained different masses of the elastomer-CTAB sample. Diluted RBCs suspensions (200 μL) treated with deionized water (1 mL) and PBS (1 mL) were used as positive and negative controls, respectively. The resultant mixtures were incubated at 37° C. for 2 h, and then centrifuged at 3,000 rpm for 10 min. All the obtained supernatant samples were transferred to a 96-well plate to measure the absorbance at 570 nm using Biotek ELX800. The hemolysis ratio was calculated using Equation 1.

$$Hemolysis Ratio = \frac{{A}_{t} - {A}_{n}}{{A}_{p} - {A}_{n}} \times 100 \% , \qquad ( Equation 1 )$$

where At, Ap and An were the absorbance values of experimental groups, positive group, and negative group, respectively.

### In Vivo Toxicity Evaluation

The skin of the 6-7 weeks old female BALB/c mice, obtained from the animal laboratory center of Guangdong province, was cut open and then sewn back after the elastomer-CTAB sample was implanted under it (n=3). The mice were sacrificed after 10 days. The main organs (heart, liver, spleen, lung, kidney, and skin) were collected, and then H&E staining analysis was performed to assess the toxicity of PUIDE-CTAB. At the same time, the hematology parameters in serum were harvested for blood routine tests (anticoagulated by EDTAK2) and blood biochemistry. Mice with no treatment were used as a control. All relevant animal experiments were approved by the Institutional Animal Care and Use Committee rules (IACUC).

### PUIDE-CTAB

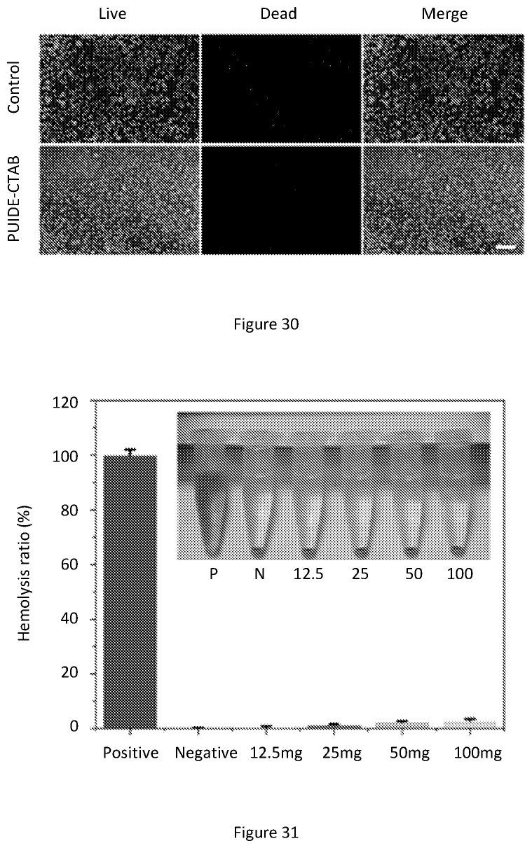

A live/dead cell viability assay was conducted to measure the effect of PUIDE-CTAB on L02 cells viability after being co-cultured for 48 h (FIG. 30). Gratifyingly, almost all cells had a strong green signal after 48 h of incubation with PUIDE-CTAB and normal spindle-like morphology, suggesting that the elastomer has no effect on biological growth in long-term biological applications. A hemocompatibility test was carried out by incubating different amounts of PUIDE-CTAB with phosphate-buffered saline (PBS) containing 2% v/v red blood cells (RBCs). The macroscopical color of all the elastomer groups, positive control group (deionized water), and negative control group (PBS) are shown in the inset of FIG. 31. Apparently, all the elastomer groups were light pink, as was the negative control group, but distinctly different from the positive control group, which was bright red. Moreover, the hemolysis ratios of all polymer groups were <3% (for 100 mg PUIDE-CTAB, the ratio was 2.83%), indicating its excellent hemocompatibility.

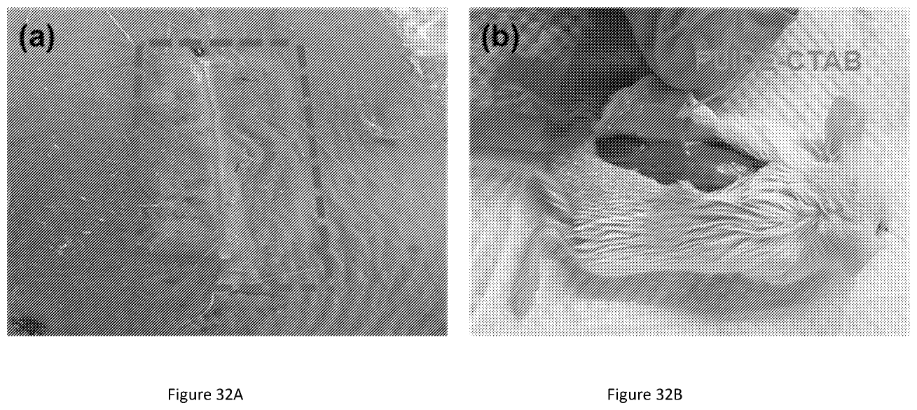

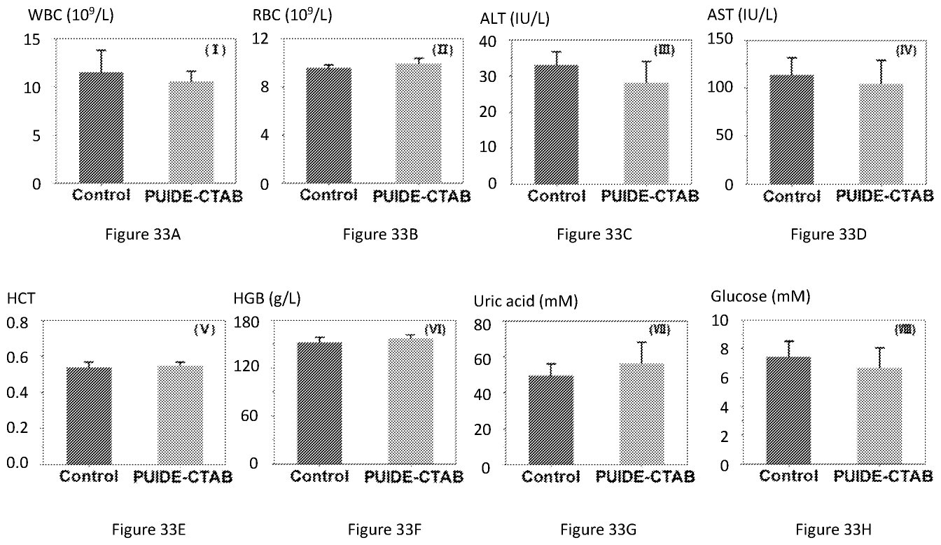

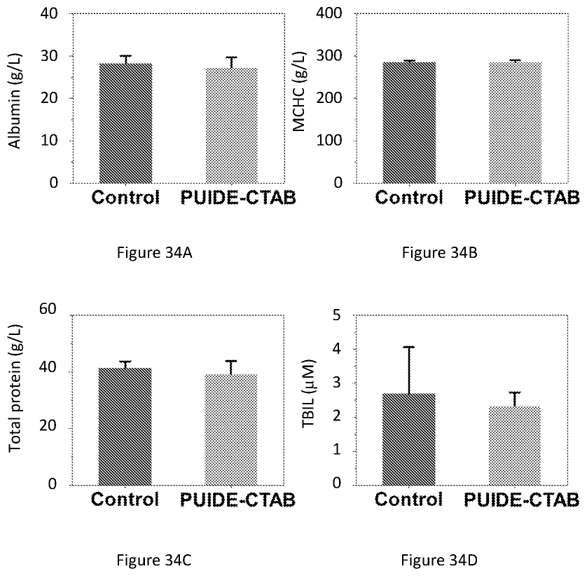

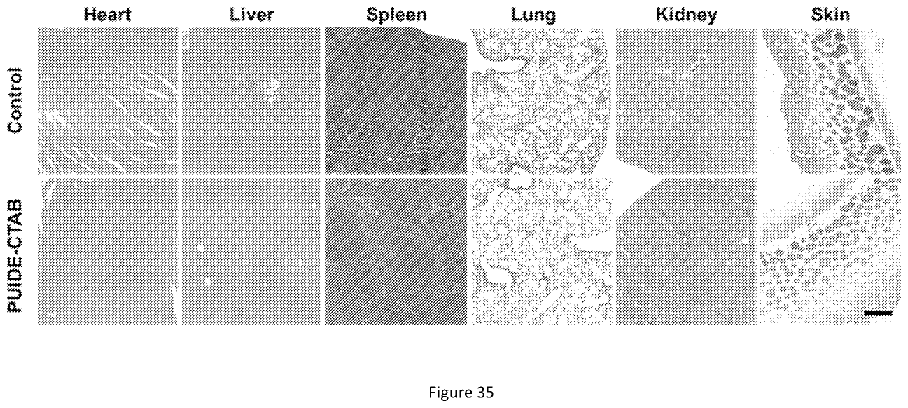

For in-vivo biocompatibility tests, one PUIDE-CTAB sample was implanted under the skin of a mouse for 10 days (FIGS. 32A-32B). Thereafter, the hematology parameters in serum were studied and compared, including white blood cell (WBC), red blood cell (RBC), alanine transaminase (ALT), aspartate transaminase (AST), hematocrit (HCT), hemoglobin (HGB), and other blood biochemical parameters. FIGS. 33A-33H and FIGS. 34A-34D show that there were no notable differences between the control and experimental groups. In addition, H&E staining was used to investigate whether there was any damage to the main organs (heart, liver, spleen, lung, kidney, and skin). PUIDE-CATB appeared not to have caused any tissue defects (FIG. 35). These results show that PUIDE-CTAB is a biocompatible elastomer with a definite potential for in-vivo medical applications.

### PUIDS-1-CTAB

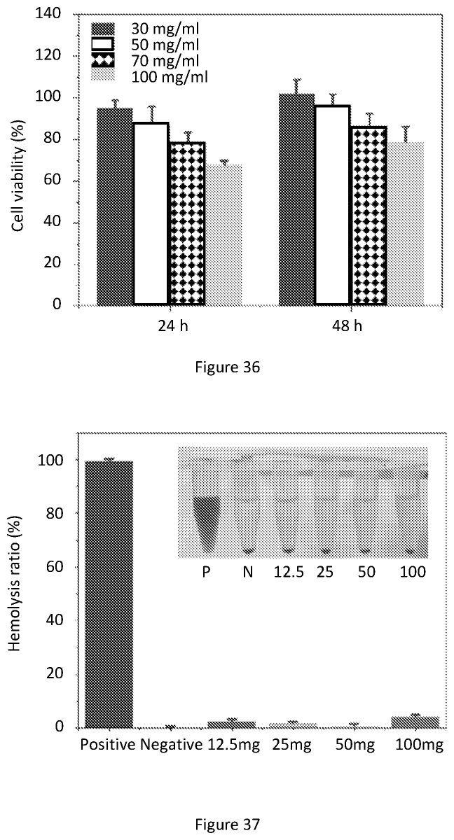

FIG. 36 shows cell toxicity evaluation of PUIDS-1-CTAB at concentrations ranging from 30 to 100 mg/mL on human L02 cells for 24 h and 48 h. The viability values of L02 cells in the presence of PUIDS-1-CTAB at different concentrations were higher than 80%, and the cell viability increased with the culture time, indicating a good cytocompatibility of PUIDS-1-CTAB. Hemolysis evaluation of the mouse red blood cells, as showed in FIG. 37, was carried out by incubating different amounts of PUIDS-1-CTAB with phosphate-buffered saline (PBS) containing 2% v/v of red blood cells (RBCs). Apparently, all the polymer groups exhibited light pink similar to the negative control group, but distinct from the positive control group, which was bright red. Moreover, the hemolysis ratios of all polymer groups were less than 5% (for 100 mg PUIDS-1-CTAB %, the ratio was 4.3%), indicating the excellent hemocompatibility of the polymer and its potential for medical applications in vivo.

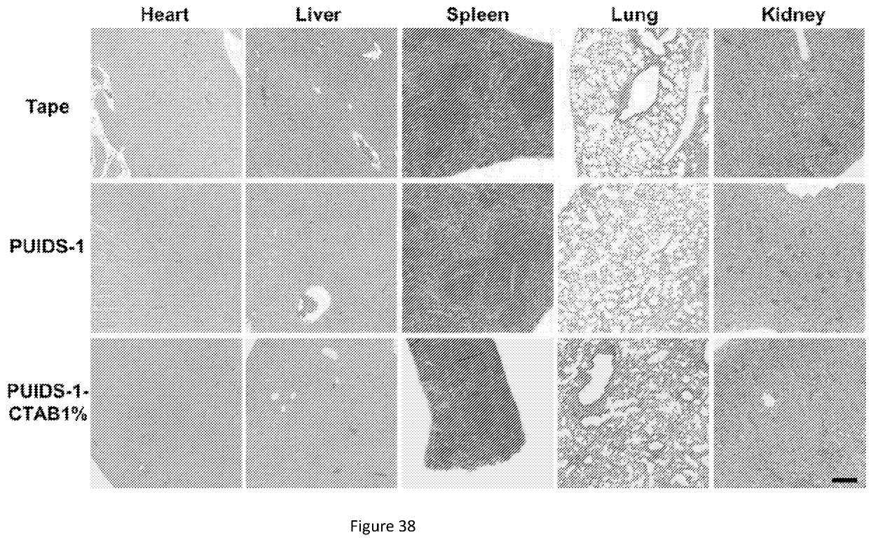

FIG. 38 shows in-vivo biocompatibility tests of PUIDS-1-CTAB. H&E staining showed that compared with control groups, there were no abnormal defects or damage in the PUIDS-1-CTAB group, suggesting the great in vivo biocompatibility of the polymer.

### Example 8—Antibacterial Activity of PUIDE-CTAB and PUIDS-CTAB Antibacterial Assays

The antibacterial activity of the elastomer-CTAB samples was measured in vitro against Gram-negative (Escherichia coli, E. coli) and Gram-positive bacteria (Staphylococcus aureus, S. aureus), 2 common bacteria responsible for most infections.

The E. coli and S. aureus strains were transferred to 10 mL sterilized nutritional broth and cultured with the elastomer-CTAB samples. As a positive control, the same volume of bacteria strains was cultured without polymers in the same amount of sterilized nutritional broth. Meanwhile, the sterilized nutritional broth without bacteria and polymers was used as a negative control (blank group). Materials in each group were placed in the disposable culture tube, and all the groups were cultured in a shaking incubator (37° C., 220 rpm/min) for certain hours. Then, the bacterial concentration of each group was detected by measuring the optical density value (ODV) at λ=630 nm with a Microplate Spectrophotometer (Biotek ELX808), and the bactericidal rate was evaluated using Equation 2:

$$Bactericidal Rate = ( 1 - \frac{D - {D}_{n}}{{D}_{p} - {D}_{n}} ) \times 1 \; 00 \% , \qquad ( Equation 2 )$$

where D, Dₚ and Dₙ are the density values of bacteria suspensions of experimental groups, positive group, and negative group, respectively.

### Live/Dead Staining Analysis

After co-cultured without/with the elastomer-CTAB samples for 2 h, the bacterial suspensions were extracted and subjected to centrifugation (4,000 rpm/min for 4 min), removal of supernatant and washing with sterile PBS three times. The final bacterial cells were stained using Live/Dead kit (Thermo fisher, L3224) for 15 min in the dark. Thereafter, the mixture was washed with PBS and observed under confocal microscope (Nikon, Japan) using 488/568 nm laser. The same experimental procedures were performed with E. coli and S. aureus.

### SEM Observation Assays

10 mL of bacterial suspensions was cultured with the elastomer-CTAB samples in a shaking incubator (37° C., 220 rpm/min) for 6 h. Thereafter, 2 mL of extracted bacterial suspension was centrifugation at 4,000 rpm/min for 4 min. The samples were washed with PBS buffer and then fixed using 4% glutaraldehyde for 2 h at 37° C. Fixed samples were washed with PBS again and sequentially dehydrated in ethanol solutions with increasing concentrations for 15 min. The samples were finally dried and subjected to SEM observations. The bacteria without the elastomer-CTAB treatment were utilized as the control group.

### PUIDE-CTAB

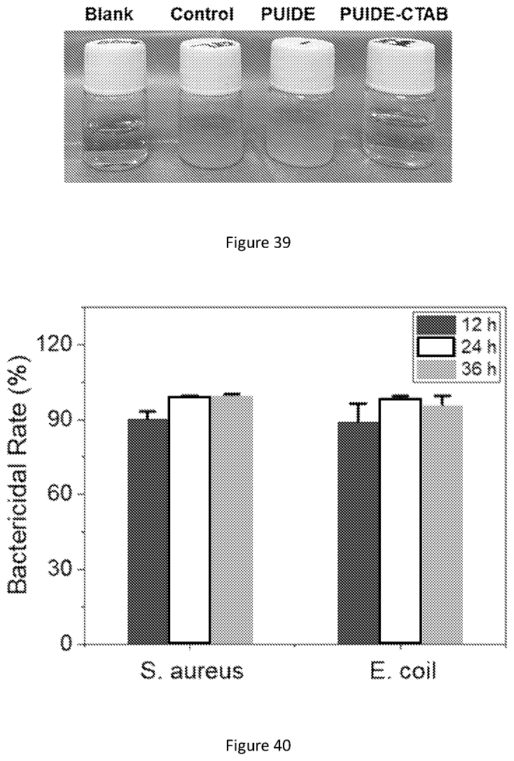

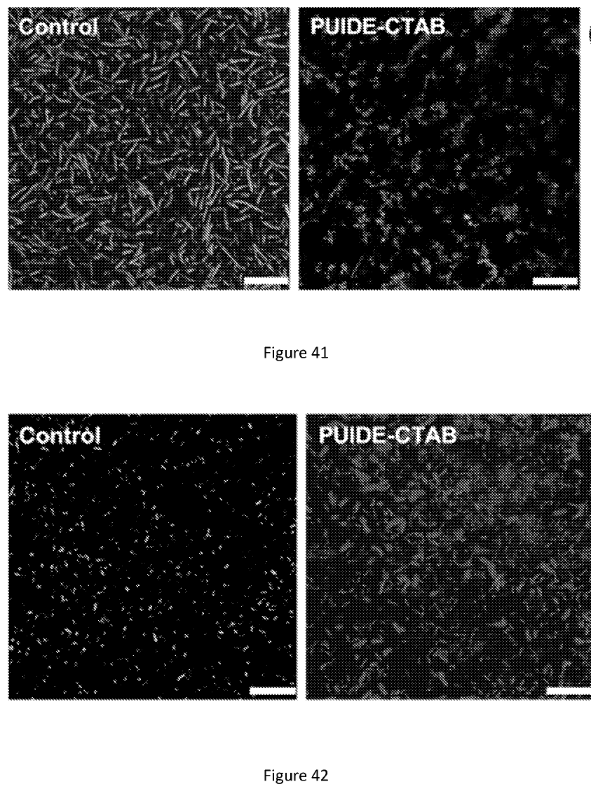

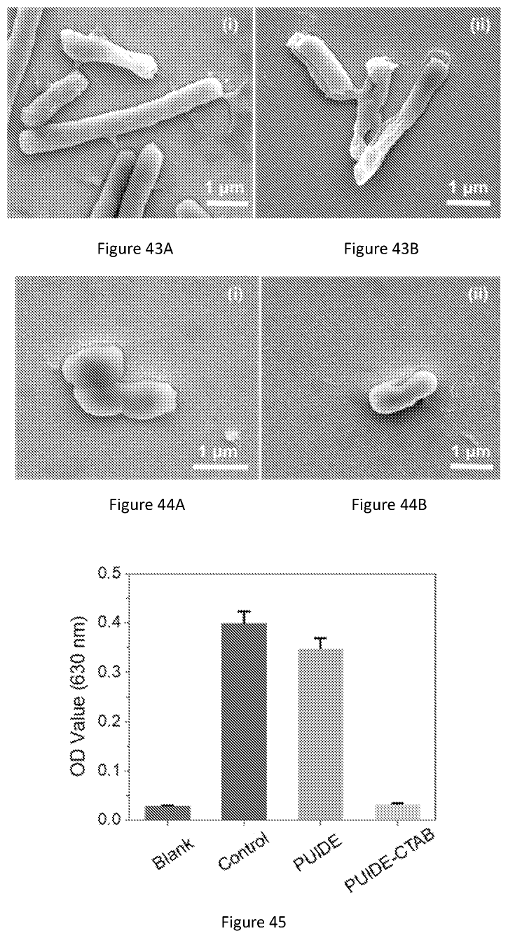

The bacterial suspensions were cultured with PUIDE-CTAB at 37° C. and extracted after different incubation durations to test the optical density values (630 nm) and determine bactericidal rates (FIG. 39). PUIDE-CTAB elastomers had a definite antibacterial property even within 12 h of culture (89.3% for E. coli and 90.4% for S. aureus), indicating effective antibacterial activity (FIG. 40). Live/dead staining of E. coli and S. aureus after incubation with the polymers for 6 h are shown in FIGS. 41 and 42. Propidium iodide (PI) is membrane impermeable and can only bind to the DNA of dead membrane-compromised cells, so the fluorescent results suggest that PUIDE-CTAB may damage the membrane integrity of E. coli and S. aureus. Therefore, scanning electron microscopy (SEM) was used to observe changes in the morphology and membrane integrity of bacteria cells after PUIDE-CTAB treatment (FIGS. 43A-43B and 44A-44B). Notably, in the absence of PUIDE-CTAB interference, the cell morphology of both E. coli and S. aureus was normal with smooth surfaces (FIGS. 43A and 44A). However, after being treated with the elastomer-CTAB, significant morphological changes and disruption were observed and the surface of the bacteria became wrinkled and incomplete, indicating that the elastomer has a significant influence on membrane integrity (FIGS. 43B and 44B). Without wishing to being bound by theory or mechanism of action, it is contemplated that the effective antibacterial activity of the elastomer-CTAB films is mainly because CTAB has many positive charges, which promotes preferential attachment of cationic copolymers to the negatively charged bacteria through electrostatic adherence, leading to the efficient destruction of cell transmembrane potential, eventually resulting in the death of bacteria. To further verify that the antibacterial activity of the PUIDE-CTAB was derived from CTAB, an E. coli suspension was cultured with PUIDE alone for 24 h. The optical density value of the PUIDE-treated group was much higher than that of the PUIDE-CTAB group, indicating that PUIDE had no antibacterial activity. Therefore, it is concluded that the antibacterial activity of the PUIDE-CTAB film is indeed related to CTAB (FIG. 45).

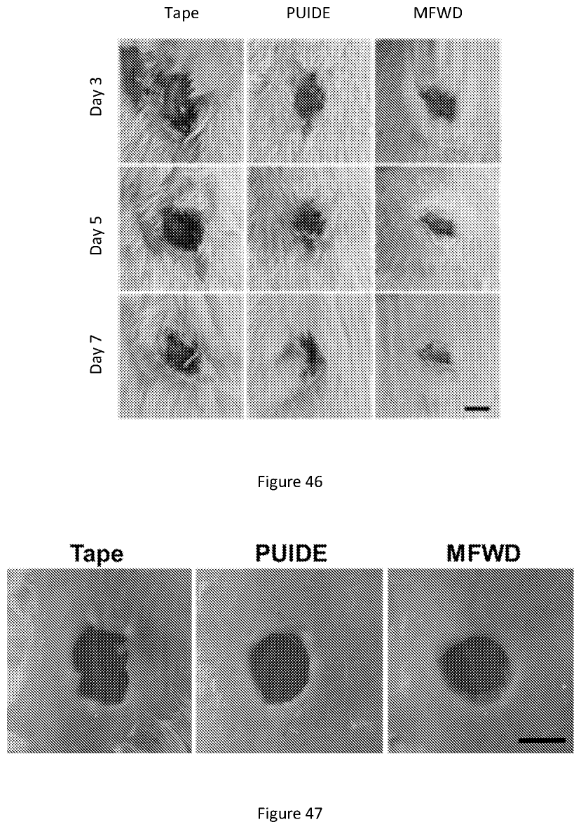

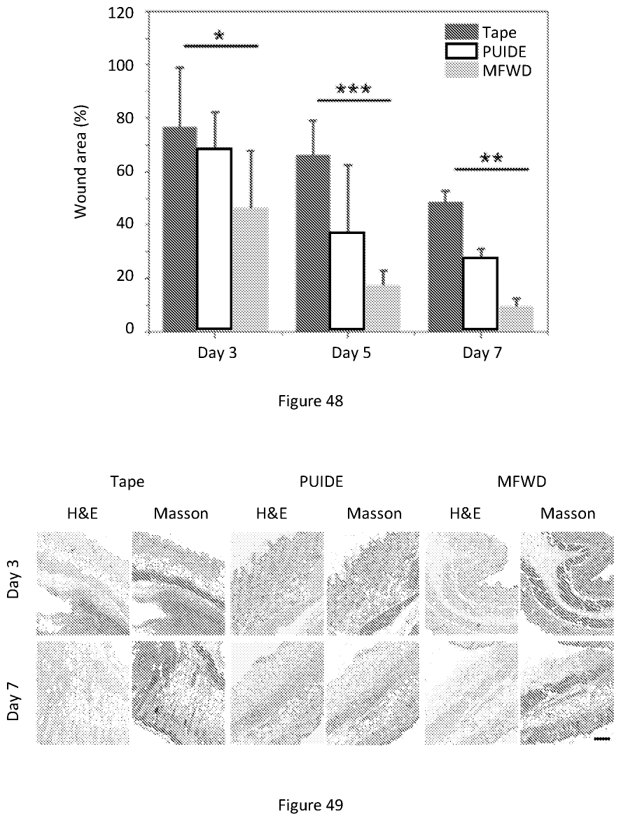

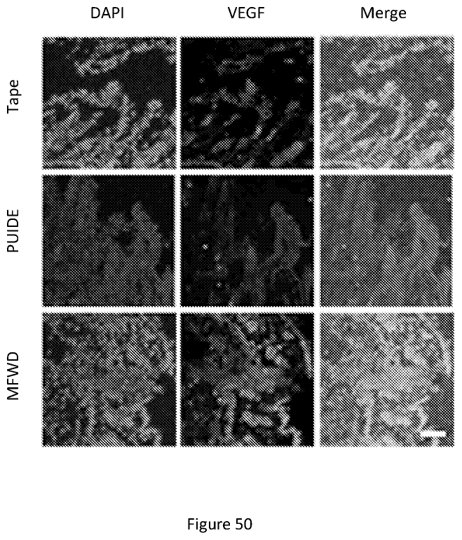

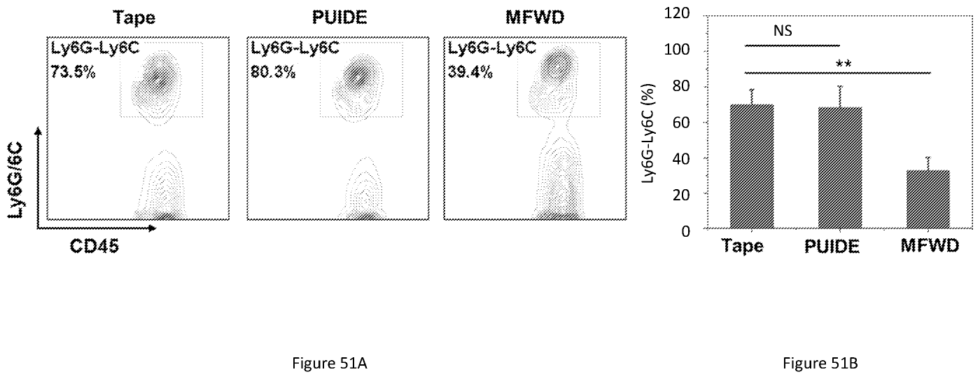

To examine the effect of the antibacterial activity of PUIDE-CTAB in promoting the healing of infected wounds, a S. aureus-infectious skin defect model was used in comparison with medical tape and PUIDE-treated groups (FIGS. 46 and 47). After 3 days of treatment, the infected skin wound area treated with the PUIDE-CTAB film was significantly reduced. Quantitative analysis of the wound area (FIG. 48) showed that after 7 days the average wound area of PUIDE-CTAB group was only 9.9±2.5%, whereas the average wound area of medical tape and PUIDE groups remained high at 48.9±3.8% and 27.8±3.2%, respectively, clearly showing that PUIDE-CTAB had a much better effect in accelerating wound healing. This ability to promote wound healing compared with medical tape and PUIDE may be due to the antibacterial properties of the CTAB-added polymer, which inhibits bacterial growth while keeping the wound suitably moist and in a sterile healing environment. To assess the histopathological structures of regenerated skin wound tissue, hematoxylin and eosin (H&E) and Masson trichrome staining of wound skins collected on days 3 and 7 were used (FIG. 49). Clearly, the PUIDE-CTAB group had a weaker inflammatory cell infiltration, more fibroblast migration and a thicker granulation tissue. Collagen deposition is an important indicator of wound healing; the fibers are rendered blue with Masson staining, the relative intensity being indicative of collagen content. On both days 3 and 7, the PUIDE-CTAB group had the highest collagen deposition compared to the 2 control groups, which may in turn lead to accelerated wound tissue reconstruction and healing rate. To confirm inflammatory regulation by PUIDE-CTAB, immunofluorescence staining of vascular endothelial growth factor (VEGF) and neutrophils detection were carried out (FIGS. 50 and 51A-51B). The regenerated wound tissue of the PUIDE-CTAB group had the highest expression of VEGF, and quantitative analysis of the ratio of Ly6g-Ly6c suggested that the mice blood of the PUIDE-CTAB group had the lowest expression of neutrophil, indicating better wound healing with less inflammation.

### Example 9—Preparation of the PUIDE-CTAB-Based Multifunctional Wound Dressing Comprising a Sensor Array

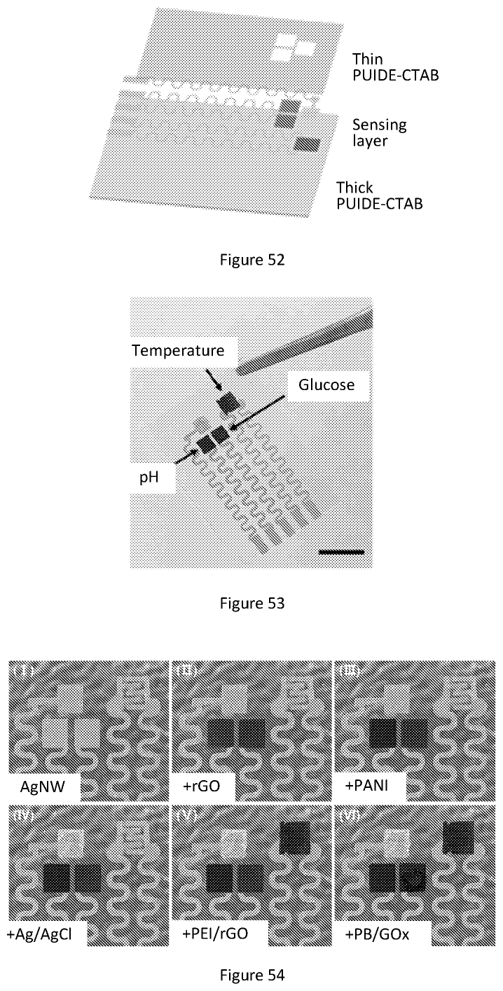

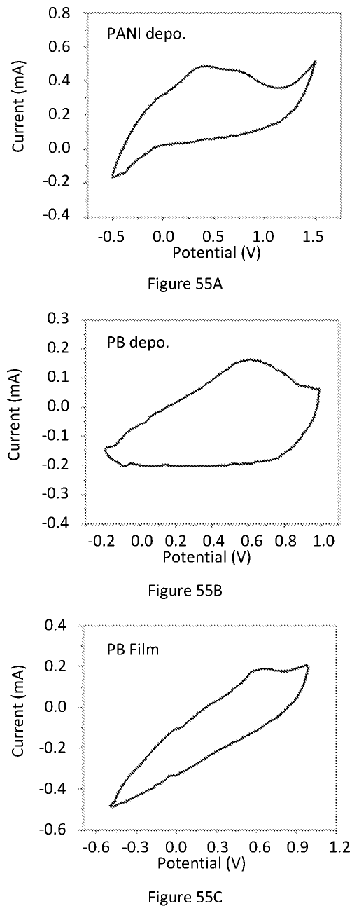

Due to the excellent mechanical properties, self-healing ability and biocompatibility of PUIDE-CTAB, a sensing modulus designed to monitor the wound-related biomarkers was prepared using said elastomer. The multifunctional wound dressing (MFWD) included 3 layers: a thick PUIDE-CTAB (as a substrate and used for sutureless wound closure), a sensing layer (glucose, pH and temperature) and a thin PUIDE-CTAB (used to prevent the direct contacting between the electrode portion outside the sensing area and the wound bed). Schematic representation of the sensing part of the MFWD and a photo thereof are shown in FIGS. 52 and 53, respectively. The serpentine electrode was fabricated from silver nanowires (AgNWs) through spray coating. Thereafter, selective and patterned functionalization of the corresponding electrodes as seen in FIG. 54 was performed such that selected functionalization of each functional material can effectively prevent cross-contamination. In this sensing system, the glucose and pH sensors were designed as a dual-electrode system containing a separate working electrode and a common Ag/AgCl reference electrode. The sensing materials on these working electrodes were Prussian blue/glucose oxidase (PB/GOx) and polyaniline (PANI), respectively, which were prepared on the corresponding electrodes by electrodeposition (FIGS. 55A-55C).

### AgNWs Electrodes Design and Manufacturing

A shadow-mask of electrode array was first designed with CorelDRAW 2019, followed by carving the shadow-mask of electrodes using Universal VLC3.60. AgNWs dispersion (10 mg/mL in isopropanol) was sprayed through the shadow-mask on a slightly modified silicon wafer, prepared by treatment with oxygen plasma and then immersion in a solution of hexyltrichlorosilane in toluene for 1 min. PUIDE-CTAB solution in chloroform was drop-casted on the AgNWs electrodes and then peeled off after the solution completely evaporated.

### Selective Functionalization of the Electrodes

All electrochemical modifications were performed with an electrochemical workstation (Keithley 2460-EC). The selective functionalization procedure of the sensor array included the following main steps, as shown in FIG. 54.

rGO modification. Before the selective functionalization, rGO solution (5 mg/mL in isopropanol) was sprayed on the specified electrodes through a shadow-mask to prevent the AgNWs electrodes from oxidation during the following electrodeposition.

PANI electrodeposition. 0.1 M aniline in 1 M HCl was prepared. The rGO modified electrode was dipped into the aniline solution and the potential was swept from −0.5 V to 1.5 V versus a commercial calomel electrode at a scan rate of 100 mV/s for 40 cycles. The remaining part of the electrodes was protected using a tape-made mask.

Ag/AgCl modification. Commercial Ag/AgCl paste was drop-casted onto the reference electrode of the pH sensor and glucose sensor. Thereafter, the sensor patch was placed in a vacuum at room temperature to completely evaporate the solvent in the Ag/AgCl.

PEI and rGO functionalization. The PEI and rGO aqueous solution (5 mg/mL in isopropanol) was coated in a specific area of the interdigital electrode by spraying. The device was left to dry in a vacuum oven at room temperature overnight. During the spray-coating, the remaining part of the electrodes was protected using a tape-made mask. The whole fabrication process was based on a previously reported method (Liu et al. Advanced Material Technology 4 (2019): 1800594).

Prussian blue (PB) electrodeposition. An electrodeposition solution of 100 mM KCl, 5 mM FeCl₃, and 5 mM K₃[Fe(CN)₆] in 10 mM HCl was prepared. The rGO modified electrode was dipped into the above solution and the potential was swept from −0.2 V to 1 V versus a commercial calomel electrode at a scan rate of 100 mV/s for 4 cycles. The remaining part of the electrodes was protected using a tape-made mask during the electrodeposition.

Drop-casting of GOx on PB. The glucose oxide/chitosan/carbon nanotube solution was prepared according to a previously reported method (Gao et al. Nature 529 (2016): 509). 3 μL of the above solution was drop-casted on the electrode deposited with PB. The glucose sensor was allowed to dry in the vacuum overnight at 4° C. with no light.

### Packaging of the Multifunctional Wound Dressings

The functionalized sensor array and extended AgNWs electrodes were encapsulated with a thin PUIDE-CTAB film with specific opening position, thus preventing the electrode portion outside the sensing area from directly contacting the wound bed.

### Example 10—In Vitro Assessment of the PUIDE-CTAB-Based Multifunctional Wound Dressing Comprising a Sensor Array

The MFWD could monitor minimal physiological changes at or around the wound sites, providing information that enables to determine the severity of infection and prevent wound degradation. Given the complexity of a healing or an infection process, the performance of sensors for glucose, pH and temperature were investigated to ensure accurate and reliable wound monitoring.

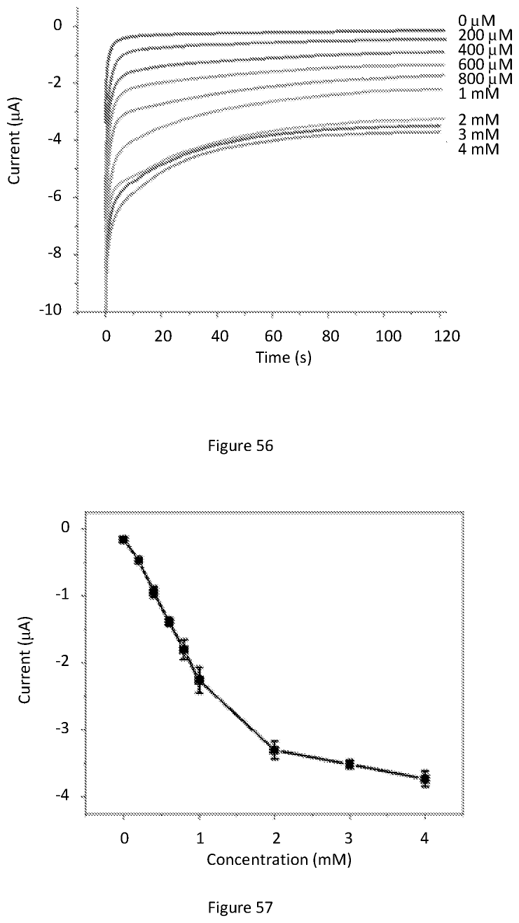

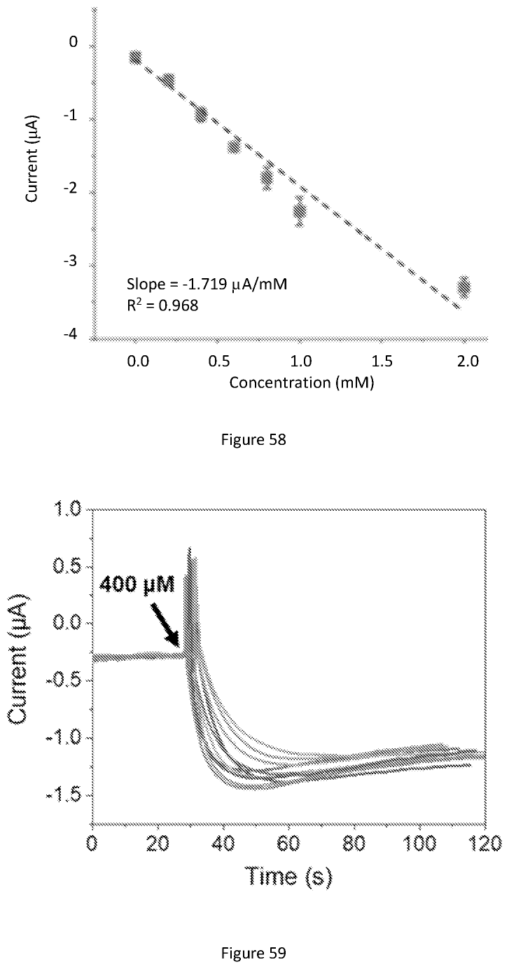

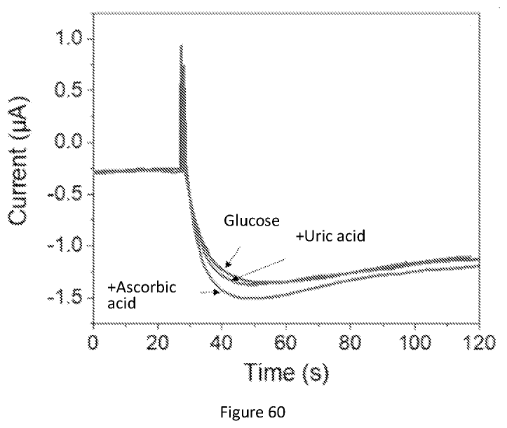

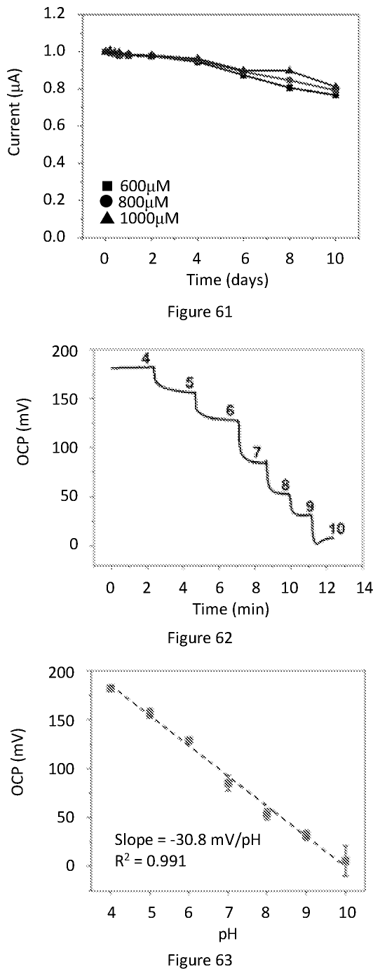

The glucose sensor was calibrated in a concentration range between 200 μM and 4 mM (FIGS. 56 and 57), which correspond to the concentrations of glucose in the typical wound milieu of chronic and healing wounds. In the range of 0-2 mM, this glucose sensor exhibited excellent linearity; the sensitivity coefficient (SC) calculated from the slope of the linear fitting curve was equal to −1.72 μA/mM, where the regression coefficient (r²) was 0.968 (FIG. 58). The glucose sensor was stable after 10 times successive tests, demonstrating good repeatability (FIG. 59). In terms of the selectivity, the sensor reacted specifically to glucose in the presence of several potentially interfering substances in the wound milieu, including uric acid and ascorbic acid (FIG. 60). It could remain active for several days (FIG. 61) and was stored at 4° C. in the dark when not in use.

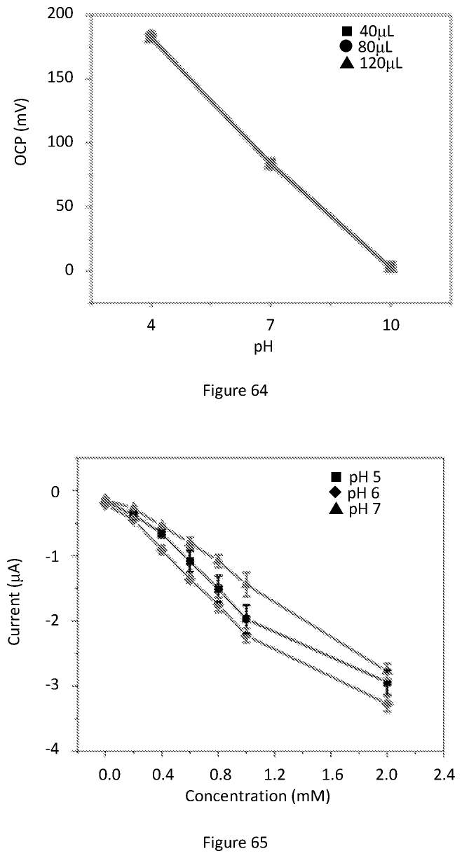

The pH sensors were calibrated under ambient conditions using real-time open current potential (OCP) measurements. FIG. 62 shows the OCP vs. time profile of the pH sensor with pH changing from 4 to 10, which covers the relevant pH range of exudate from an infected wound. This pH sensor showed an excellent linear response, with a SC and r² of −30.8 mV/pH and 0.991, respectively (FIG. 63). The volume of the liquid had no effect on the performance of the pH sensor (FIG. 64). Since the efficiency of GOx and thereby the sensitivity of glucose sensor could be affected by pH changes, the pH sensor was also used to calibrate the pH-dependent deviation of GOx-based glucose sensors (FIG. 65) to ensure accuracy during practical applications. Therefore, the calibrated pH sensor with high sensitivity and reliability showed promising potential for monitoring pH variations at wound sites.

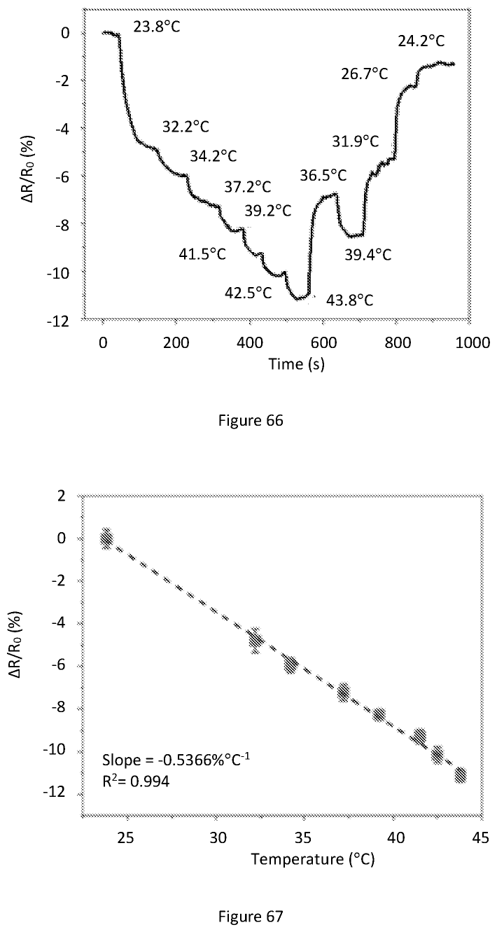

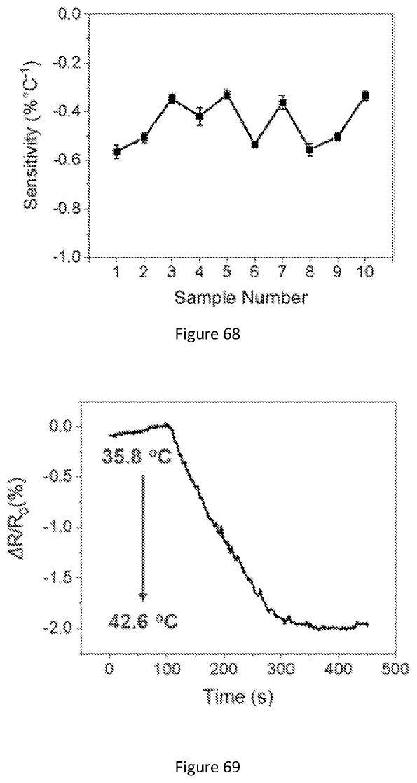

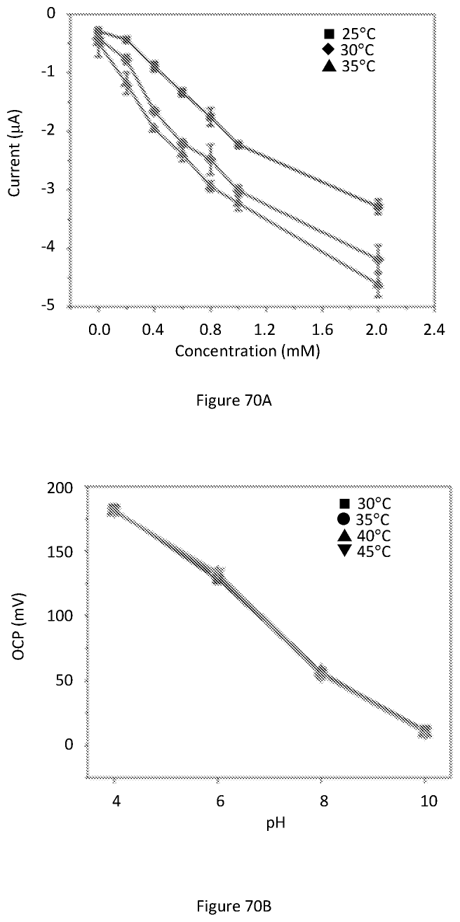

Temperature sensor performance when immersed in water was monitored by the resistance of the sensor when the temperature of the water was changed. It responded rapidly and reliably in real-time to temperature variations ranging from 23.8 to 43.8° C. (FIG. 66). There was an excellent linear relationship between the response of these temperature sensors and temperature changes, with the SC and r² readings being ˜−0.537%° C.⁻¹ and 0.994, respectively, through the linear fitting equation (FIG. 67). In addition, there was no significant difference in the sensitivity of 10 temperature sensors, indicating their reliable repeatability (FIG. 68). The dynamic response of the temperature sensor was also analyzed by changing the temperature from 35.8 to 42.6° C. (a physiological temperature range); it displayed a fast and reliable response (FIG. 69), demonstrating the temperature sensor as a potential candidate for wound temperature monitoring. The temperature dependency of the glucose and pH sensors were subsequently investigated. The sensitivity of the glucose sensor increased with increasing temperature over a certain range, whereas the sensitivity of the pH sensor basically remained constant under different temperature conditions (FIGS. 70A and 70B). Therefore, these calibrations show that wound-related biomarkers at or around a wound site can be accurately monitored in real-time.

### Example 11—Sutureless Wound Closure and Infected Wound Monitoring Using MFWD

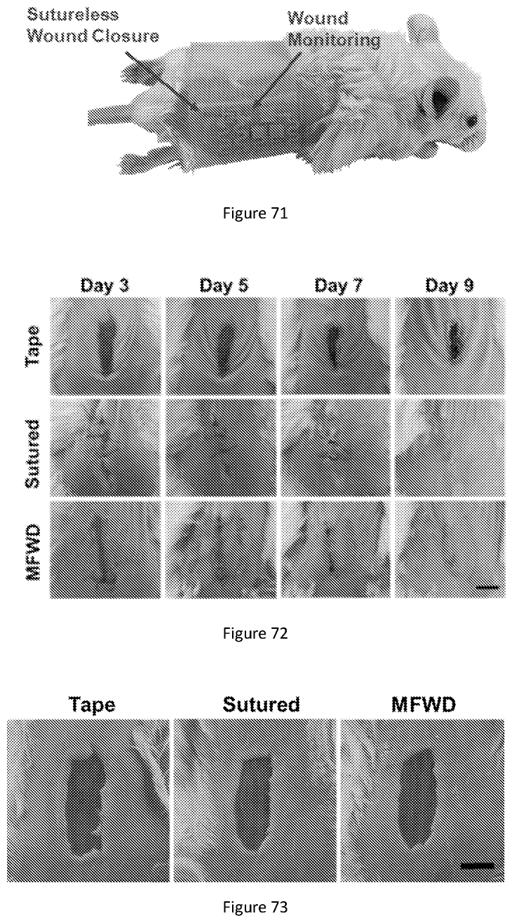

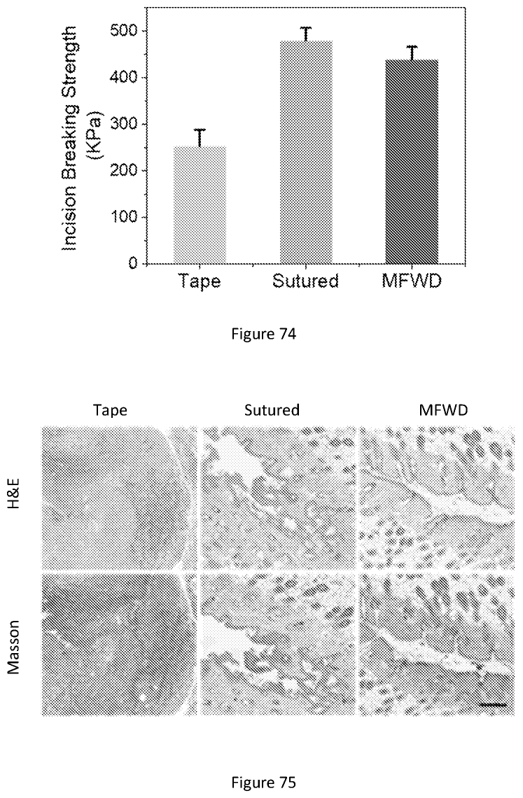

The above results demonstrated that MFWD has excellent self-healing properties, antibacterial activity and biocompatibility, all beneficial in healing infected wounds. To estimate the sutureless wound closure and infected wound monitoring behavior of MFWD, the closure rate and wound related parameters (temperature, pH, and glucose) were recorded during healing. FIG. 71 shows a schematic diagram of the in-situ animal studies. The sutureless wound closure ability of MFWD was tested using a full-thickness skin incision model. The incisions created on the backs of mice were sealed by suture and MFWD, and the incisions covered with tape were used as the control group. After 9 days, the tape-treated skin incisions obviously had a partially retained gap, whereas there was no gap in the groups treated with suture and MFWD (FIGS. 72 and 73), i.e., the suture and MFWD-treated skin incisions had healed. The incision breaking strength was also measured. FIG. 74 shows that the breaking strength of MFWD-treated skin incisions was almost the same as the suture-treated groups but was much higher than that of the tape-treated groups, demonstrating that MFWD could effectively close the wound and promote wound healing in a sutureless way. Histological analysis showed that both the suture and MFWD-treated groups had almost intact and thickened dermal tissue, and some skin appendages (e.g., hair follicles) could be seen, indicating that the wound was almost completely healed (FIG. 75). Masson staining also showed that dense and well-organized collagen fibers were deposited around incisions treated by MFWD.

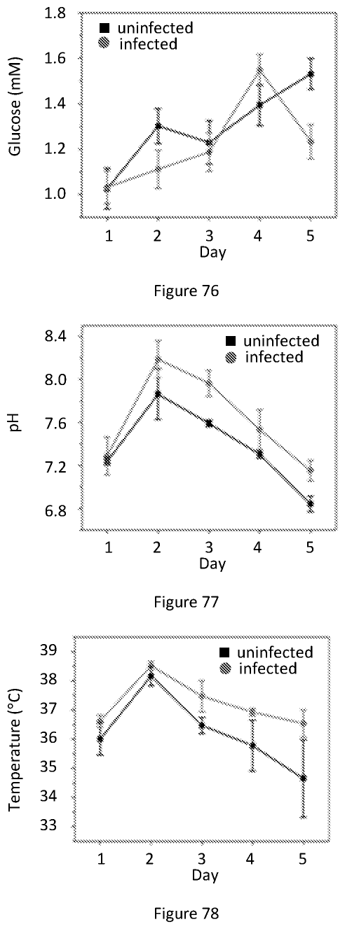

To explore changes in relevant parameters during the infected/uninfected wound healing, the glucose, pH, and temperature of the wounds with/without bacterial infections were measured (FIGS. 76-78 and Table 2). On the second day, from the recorded results, it was found that there were significant differences in these physiological parameters between the infected and uninfected wounds. According to previous studies, the glucose concentration of infected wounds will be lower than that of normal healing wounds and infections will increase the temperature and change the pH from acidic to alkaline. However, increased temperature and pH in the group of uninfected wounds may be caused by the normal inflammatory response at the wound site. It was also found that the temperature and pH values of uninfected wounds decline significantly faster after the second day as compared to those of infected wounds, indicating the better healing status of the former. Although these physiological parameters of the infected wounds were higher than those of uninfected wounds, they nevertheless showed a decreasing trend, suggesting that infected wounds were also gradually healing, which may be closely related to the antibacterial properties of MFWD. All the above results demonstrate that in addition to the sutureless wound closure ability, MFWD could precisely monitor the wound status by detecting wound-related parameters, and efficiently promoting the healing of infected wounds.

TABLE 2
The calibrated values of temperature, pH, and glucose
of the wounds with different treatments at day 5.

| Day 5 | Example | Temperature (° C.) | pH | Glucose (μM) |
|---|---|---|---|---|
| Uninfected | 1 | 35.99751 | 6.92251 | 1.55334 |
| | 2 | 33.32971 | 6.78341 | 1.58554 |
| | 3 | 34.62177 | 6.8216 | 1.45499 |
| Infected | 1 | 36.0828 | 7.26324 | 1.23454 |
| | 2 | 36.50535 | 7.10875 | 1.15699 |
| | 3 | 37.02525 | 7.0885 | 1.30712 |

### Comparative Example—Elastomers Based on an Aromatic Disulfide

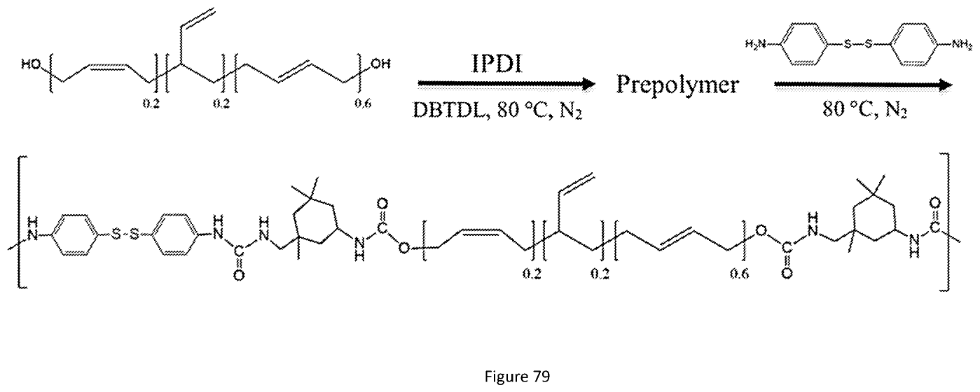

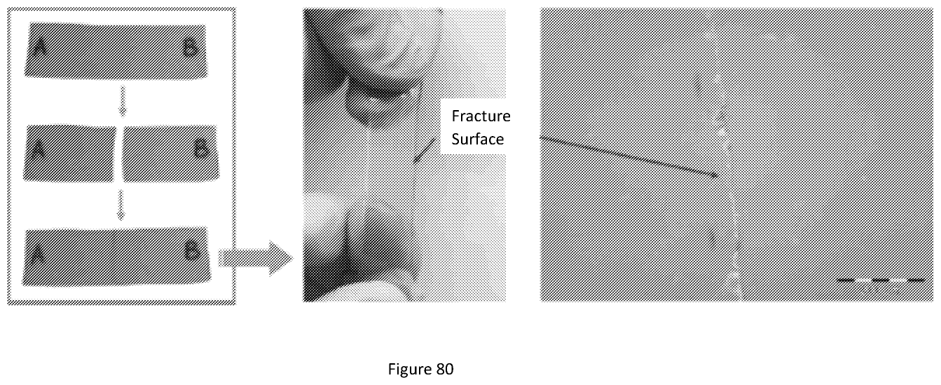

In a comparative study, a self-healing polybutadiene polyurea urethane elastomer based on an aromatic disulfide dynamic linkages (PBPUU) was prepared and studied for its mechanical properties and biocompatibility. The synthesis process and the obtained molecular structure of the elastomer are shown in FIG. 79. FIG. 80 shows that the polymer could recover to its elastic property after a complete cut, demonstrating the highly efficient self-healing property of PBPUU. However, the in vitro biocompatibility results showed that the human L02 cells were dead when cultured with PBPUU, which suggests that the PBPUU polymer was toxic to cells and cannot be used as a dressing material for human wound treatment.

It is appreciated by persons skilled in the art that the present invention is not limited by what has been particularly shown and described hereinabove. Rather the scope of the present invention includes both combinations and sub-combinations of various features described hereinabove as well as variations and modifications. Therefore, the invention is not to be constructed as restricted to the particularly described embodiments, and the scope and concept of the invention will be more readily understood by references to the claims, which follow.

1-47. (canceled)

48. A self-healing biocompatible elastomer comprising polymeric chains comprising units of formula (A1) 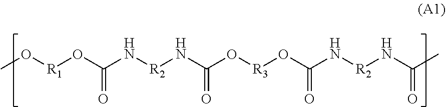
wherein
R₁ is selected from the group consisting of a linear (C₂-C₂₀)alkylene and R₄—S—S—R₄′—, wherein R₄ and R₄′ are independently a linear (C₁-C₁₀)alkylene;
R₂ is a linear or cyclic (C₄-C₁₀)alkylene; and
R₃ is selected from the group consisting of a polybutadiene, a polybutene, a polyethylene, a polypropylene, and a polyisoprene.

49. The elastomer according to claim 48, wherein R₁ is a linear (C₂-C₂₀)alkylene; or wherein R₁ is a linear C₁₀ alkylene.

50. The elastomer according to claim 48, wherein R₁ is R₄—S—S—R₄′.

51. The elastomer according to claim 50, wherein each one of R₄ and R₄′ is a C₂ alkylene, and/or wherein the S—S content of the elastomer is up to about 3% (w/w).

52. The elastomer according to claim 48, wherein R₂ is selected from the group consisting of butylene, hexylene, cyclohexylene, and decylene; or wherein R₂ is represented by formula (D1)

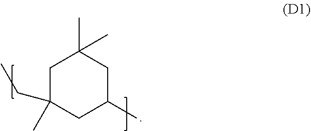

53. The elastomer according to claim 48, wherein R₃ is a polybutadiene.

54. The elastomer according to claim 53, wherein the polybutadiene comprises 1,3-butadiene derived-monomer units of formula (B1), formula (B2), and formula (B3), 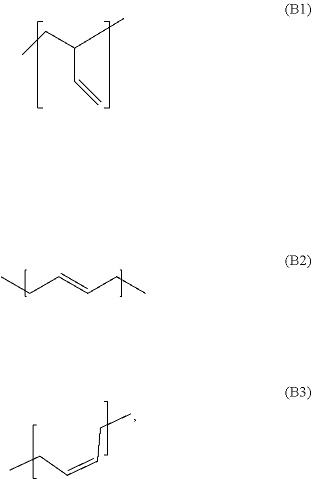
wherein the proportion of the monomer unit of formula (B1) is 10 to 60 mole percent, the proportion of the monomer unit of formula (B2) is 20 to 70 mole percent, and the proportion of the monomer unit of formula (B3) is 10 to 50 mole percent in the entirety of the 1,3-butadiene-derived monomer units present in one unit of formula (A1); and/or wherein the polybutadiene comprises about 20 mole percent monomer units of formula (B1), 60 mole percent monomer units of formula (B2), and 20 mole percent monomer units of formula (B3) in the entirety of the 1,3-butadiene-derived monomer units present in one unit of formula (A1).

55. The elastomer according to claim 48, wherein the unit of formula (A1) is:

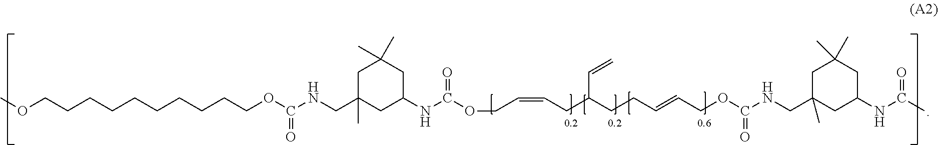

56. The elastomer according to claim 55, wherein the elastomer has the structure of formula (A3): 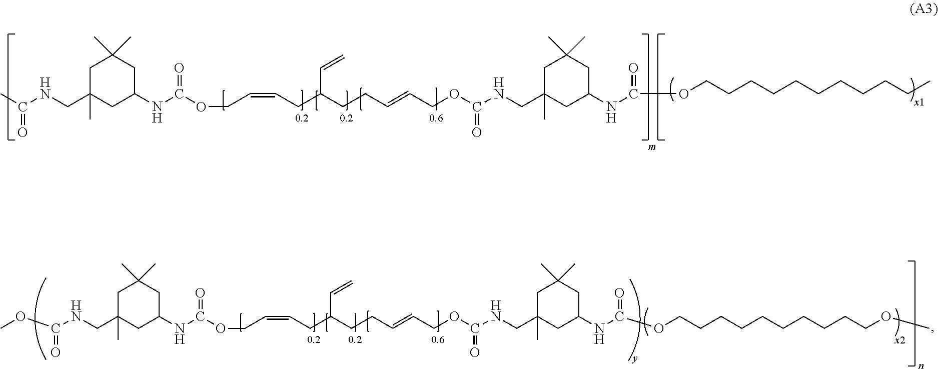
wherein 0≤y<(x1+x2), m ranges between 1 and 1000, n ranges between 1 and 1000, y ranges between 1 and 100, x1 ranges between 1 and 100, and x2 ranges between 1 and 100.

57. The elastomer according to claim 48, wherein the unit of formula (A1) is:

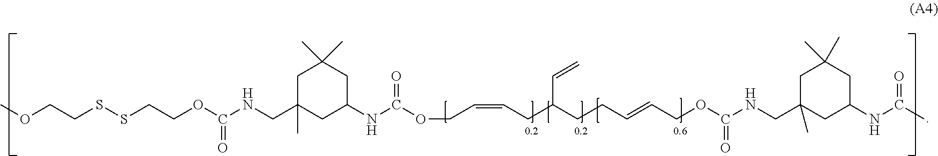

58. The elastomer according to claim 57, wherein the elastomer has the structure of formula (A5): 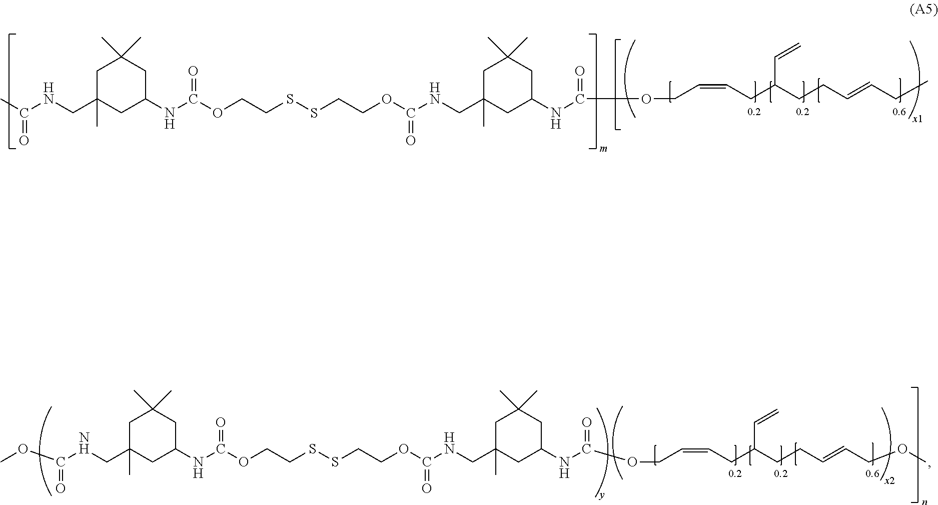
wherein 0≤y<(x1+x2), m ranges between 1 and 1000, n ranges between 1 and 1000, y ranges between 1 and 100, x1 ranges between 1 and 100, and x2 ranges between 1 and 100.

59. A one-pot method for preparing a self-healing biocompatible elastomer, the method comprising reacting a hydroxyl-terminated polybutadiene (HTPB) with a linear or cyclic (C₄-C₁₀)alkylene diisocyanate compound and a hydroxyl-terminated compound selected from a linear (C₂-C₂₀)diol and a hydroxyl-terminated linear (C₁-C₁₀) alkyl disulfide.

60. The method according to claim 59, wherein the hydroxyl-terminated compound is a linear (C₂-C₂₀)diol; or wherein the hydroxyl-terminated compound is 1,10-decanediol; or wherein the hydroxyl-terminated compound is a hydroxyl-terminated linear (C₁-C₁₀) alkyl disulfide; or wherein the hydroxyl-terminated compound is 2-hydroxyethyl disulfide.

61. The method according to claim 59, wherein the diisocyanate compound is selected from the group consisting of isophorone diisocyanate (IPDI), 1,4-tetramethylene diisocyanate, 1,6-hexamethylene diisocyanate (HDI), and 1,10-decamethylene diisocyanate; and/or wherein the diisocyanate compound is IPDI.

62. The method according to claim 59, wherein the HTPB comprises 1,3-butadiene derived-monomer units of formula (B1), formula (B2), and formula (B3), wherein the proportion of the monomer unit of formula (B1) is 10 to 60 mole percent, the proportion of the monomer unit of formula (B2) is 20 to 70 mole percent, and the proportion of the monomer unit of formula (B3) is 10 to 50 mole percent in the entirety of the 1,3-butadiene-derived monomer units present in the HTPB; and/or wherein the HTPB comprises about 20 mole percent monomer units of formula (B1), 60 mole percent monomer units of formula (B2), and 20 mole percent monomer units of formula (B3).

63. The method according to claim 59, wherein the molar ratio between the HTPB, the hydroxyl-terminated compound, and the linear or cyclic (C₄-C₁₀)alkylene diisocyanate compound is about 1:1:2.1.

64. An elastomer obtained by the method according to claim 59.

65. An antibacterial composition comprising the elastomer according to claim 48, and a quaternary ammonium compound.

66. The antibacterial composition according to claim 65, wherein the quaternary ammonium compound is cetyltrimethylammonium bromide (CTAB); and/or wherein the quaternary ammonium compound is present in the composition in a weight percentage of up to about 1% of the total weight of the composition.

67. The antibacterial composition according to claim 65, being in a form of a film.

68. A method for the preparation of an antibacterial composition comprising mixing the elastomer according to claim 48, a quaternary ammonium compound and a solvent to form a homogeneous mixture and evaporating the solvent.

69. A wound dressing comprising a film made of the elastomer according to claim 48.

70. The wound dressing according to claim 69, further comprising at least one sensor for the detection of one or more parameters of the wound, wherein the at least one sensor is embedded within or deposited onto the film.

71. The wound dressing according to claim 70, wherein the at least one sensor is selected from the group consisting of a glucose sensor, a pH sensor, and a temperature sensor; and/or wherein the at least one sensor comprises an electrode and a sensing layer disposed on a portion of said electrode and/or electrically connected thereto, and optionally, a reference electrode.

72. The wound dressing according to claim 71, wherein the electrode is made of a micro-sized or nanosized conductive material embedded within or deposited onto the film; and/or wherein the conductive material is selected from the group consisting of a metal, a metal alloy, a metal carbide, a metal nitride, a metal oxide, a metal silicide, carbon, a polymer, ceramics, and combinations thereof and/or wherein the conductive material has a form selected from the group consisting of nanoparticles, nanowires, nanotubes, nanoflakes, nanofibers, nanoribbons, nano-whiskers, nanostrips, nanorods, and combinations thereof.

73. The wound dressing according to claim 71, wherein the sensing layer comprises a material selected from the group consisting of a biorecognition element, a redox-active element, an electrically conducting material, a thermally conductive material, and any combination thereof; and/or wherein the sensing layer comprises a material selected from the group consisting of polyethyleneimine (PEI), glucose oxidase (GOx), carbon nanotubes, reduced graphene oxide (rGO), polyaniline (PANI), K₃[Fe(CN)₆] (Prussian blue), and any combination thereof.

74. The wound dressing according to claim 70, comprising:
a glucose sensor comprising an electrode made of Ag nanowires and a sensing layer comprising Prussian blue and glucose oxidase;
a pH sensor comprising an electrode made of Ag nanowires and a sensing layer comprising PANI; and
a temperature sensor comprising an electrode made of Ag nanowires and a sensing layer comprising PEI and reduced graphene oxide; and/or
further comprising an additional film made of said elastomer or of an antibacterial composition comprising said elastomer and a quaternary ammonium compound, wherein the additional film covers at least a portion of the at least one sensor.

75. The wound dressing according to claim 69, further comprising at least one of a drug release layer, a self-cleaning protecting layer, and a wearable data processing device.

76. A method for treating and/or monitoring a condition of a wound comprising applying the wound dressing according to claim 70 to the wound.

77. The method according to claim 76, wherein the condition of the wound is monitored by the at least one sensor; and/or wherein applying the wound dressing to the wound comprises performing a surgical incision on a body part and applying the wound dressing to said body part, wherein the incision is performed atop the wound dressing.
